(19) United States
(12) Patent Application Publication
(10) Pub. No.: US 2026/0208174 A1
(43) Pub. Date: Jul. 23, 2026

(54) CATALYST SYSTEM AND METHOD FOR CARBONYLATION REACTION

(71) Applicants: Zhejiang Nhu Co., Ltd., Zhejiang (CN); Zhejiang Nhu Special Materials Co., Ltd., Zhejiang (CN); Shandong Nhu Fine Chemical Science and Technology Company Ltd., Shandong (CN)

(72) Inventors: Chuang Wang, Zhejiang (CN); Jiasheng Pan, Zhejiang (CN); Haoran LI, Zhejiang (CN); Xin Hong, Zhejiang (CN); Yuhong Zhang, Zhejiang (CN); Lei WU, Zhejiang (CN); Yong Xu, Shandong (CN); Shuoqing Zhang, Zhejiang (CN)

(21) Appl. No.: 19/143,606

(22) PCT Filed: Sep. 15, 2023
(86) PCT No.: PCT/CN2023/119152

§ 371 Date: Jun. 26, 2025

(30) Foreign Application Priority Data
Dec. 28, 2022 (CN) 202211699590.2

(51) Int. Cl. B01J 31/24; C07C 51/12

(52) U.S. Cl. CPC B01J 31/2433; B01J 31/2438; B01J 31/2476; C07C 51/12; B01J 2231/321; B01J 2531/004; B01J 2531/0241; B01J 2531/822; B01J 2531/824; B01J 2531/827

(57) ABSTRACT

A catalyst system for a carbonylation reaction and a method for a carbonylation reaction. The catalyst system includes (a) a group VIII metal or a compound of the group VIII metal; (b) a bidentate phosphine ligand; and (c) an acidic additive. The component (b) is formula (I): R¹>P-A-Ar—B-P<R² (I), wherein P represents a trivalent phosphorus atom; Ar represents an aromatic group, A and B each represent an alkylene group, and A and B are located at adjacent positions on Ar; R1 and R2, each together with P atoms linked thereto, form a phosphorus monocyclic group of which the ring contains at least three carbon atoms; the monocyclic group includes an alicyclic group having a substituent or no substituent, a lactone group, a lactam group, a cyclic ketone group, an N-containing heterocyclic group, and an O-containing heterocyclic group, and the substituent is linked to the monocyclic group.

### TECHNICAL FIELD

The present disclosure relates to a catalyst system for a carbonylation reaction and a method for an olefin carbonylation reaction using the catalyst system.

### BACKGROUND

In the past decades of chemical research, the catalyst systems containing transition metals and phosphine ligands have been widely applied in various types of reaction because of their high catalytic activity and high selectivity, for example, cross-coupling reactions (Buchwald-Hartwig C—N bond and C—O bond formation reaction, Stille reaction, Sonogashira reaction, Suzuki-Miyaura reaction, etc.), asymmetric hydrogenation reactions, and carbonylation reactions. One of the olefin carbonylation reactions, as shown in Reaction Scheme 1 below, comprises converting an unsaturated hydrocarbon such as an olefin, CO, and an alcohol into a corresponding saturated carboxylic acid ester in the presence of a metal/ligand or metal complex.

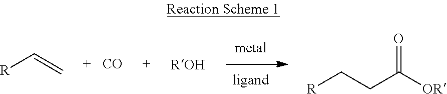

Such saturated carboxylic acid esters are an important fine chemical product and are widely applied in such fields as pharmaceuticals, resins, coatings, food solvents, plasticizers, and cosmetics. Since the discovery of the first olefin carbonylation reaction in 1938, this type of reaction has always been one of the research hotspots in the fields of organic synthesis and catalysis. In the olefin carbonylation reaction, methyl propionate as the carbonylation reaction product of ethylene is a major intermediate for preparing methyl methacrylate.

Patent Literature 1 describes a process for the carbonylation of ethylene and a catalyst system for use therein. This catalyst system comprises a metal of Group VIII or a compound thereof, and a bidentate phosphine ligand having a tertiary carbon group and an aryl bridge, where the bidentate phosphine ligand is particularly represented by bis(di-t-butyl phosphino)-o-xylene. This catalyst system exhibits better reaction rates in olefin carbonylation reactions, but it tends to deactivate during continuous operation as the palladium compound is reduced to a palladium metal, which leads to high costs and limited industrial applications.

Patent Literature 2 describes carbonylation of olefins. It also describes the catalyst system used in this carbonylation reaction, including a source of palladium cations, a source of anions, and a source of bidentate diphosphine having a structure of formula I: R¹R²P—X-P-R³R⁴ (I). Examples of the bidentate diphosphine include 1,3-bis(di-i-propylphosphino) propane, 1,3-bis(di-n-butylphosphino) propane, 1,5-bis(dimethylphosphino)-3-oxapentane, etc. The catalyst system containing the bidentate diphosphine provides substantially higher reaction rates, yields and/or selectivities to products in various monocarbonylation reactions. However, this catalyst system has the disadvantage that the ligand is prone to dissociation, resulting in catalyst poisoning and a failure to recycle the catalyst.

Patent Literature 3 describes diphosphines containing a 2-phospha-tricyclo[3.3.1.1.{3,7}]decyl group. The diphosphine is a bidentate phosphine ligand having a covalent bridging group for use in the carbonylation reaction of an unsaturated compound, which is represented by the structural formula of R¹>P-R²-PR³R⁴. A specific compound of the diphosphine is, e.g., 1,3-P,P′-di(2-phospha-1,3,5,7-tetramethyl-6,9,10-trioxatricyclo[3.3.1.1 {3,7}]decyl) propane. At the equivalent TON, this diphosphine ligand is used less as compared to the ligand described in aforementioned EP0495547A. However, the ligand exhibits low selectivity and narrow application scope of substrates, and still has room for further optimization.

Patent Literature 4 describes a bidentate phosphine ligand useful in the catalyst system. This bidentate phosphine ligand is a bidentate phosphine ligand having a phosphorane cyclic group and represented by the structural formula of R¹R²M¹-R-M²R³R⁴. In this bidentate phosphine ligand, two di-tertiary alkylphosphino groups are attached to each other via an alkylene group as a bridging group, or the phosphorane cyclic group is attached to phosphorus via secondary carbon and the alkylene group serves as a bridging group. Examples of this bidentate phosphine ligand include 2,3-bis(di-tertiary-butylphosphino) butane and 2,3-P,P′-di(2-phospha-1,3,5,7-tetramethyl-6,9,10-trioxatricyclo[3.3.1.1{3.7}decyl]butane. This bidentate phosphine ligand can provide good selectivity and reduced production of polymers in the carbonylation reactions, but its catalytic efficiency remains to be improved.

Patent Literature 5 describes a process for the carbonylation of an ethylenically unsaturated compound and a catalyst therefor. It extends the teaching of the bidentate phosphine ligand in the aforementioned U.S. Pat. No. 6,156,934A to bidentate diphosphine having a 1,2-substituted aryl bridge among the types described in the aforementioned WO96019434A1. The bidentate diphosphine has the following structural formula: R¹>P-A¹-R-A²-PR²R³ Specific examples of the bidentate diphosphine include 1,2-P,P′-di(2-phospha-1,3,5,7-tetramethyl-6,9,10-trioxatricyclo[3.3.1.1{3.7}decyl]-methylene-benzene. The catalytic efficiency of this bidentate diphosphine is somewhat improved in the reaction.

Patent Literature 6 describes a phospha-adamantane(s) catalytic system. This catalyst system could catalyze the carbonylation reaction of ethylenically unsaturated compounds. This catalyst system is a bidentate phosphine ligand with a side-substituted phospha-adamantane group. Specific examples of the bidentate phosphine ligand include 1,2-bis(diadamantylphosphinomethyl)benzene and 1,2-bis(diadamantylphosphinomethyl) naphthalene. When this catalyst system is used, the byproducts generated during the reaction are significantly reduced and the replenishment of catalyst is decreased. Nevertheless, there is a need to add a polymeric dispersant to this system, and the polymeric dispersant needs to be recovered later, which increases the operational procedures and costs.

Patent Literature 7 describes a catalyst system. It is used to catalyze the carbonylation reaction of ethylenically unsaturated compounds. This catalyst system comprises a metal of Group VIB or VIIIB or a compound thereof, an acid, and a bidentate phosphine, arsine or stibine ligand. Specific examples of the bidentate ligand include 1,2-bis(diadamantylphosphinomethyl)benzene, 1,2-bis(di-3,5-dimethyladamantylphosphinomethyl)benzene, 1,2-bis(di-5-tert-butyladamantaylphosphinomethyl)benzene, etc. The bidentate ligand is present in at least a 2:1 molar excess compared to the metal or the acid is present in at least a 2:1 molar excess compared to the ligand. In the process described in this patent, a polymeric dispersant is also used. Besides, the acid is used in a large amount in this process, which directly increases the cost.

Patent Literature 8 describes carbonylation of ethylenically unsaturated compounds. The catalyst system used therein comprises a metal of Group 8, 9 or 10 or a compound thereof, and a bidentate phosphine ligand bridged by a non-aromatic cyclic hydrocarbon structure. Specific examples of the bidentate ligand phosphine include cis-1,2-bis(di-tert-butylphosphinomethyl)-4,5-dimethylcyclohexane, cis-1,2-bis(di-tert-butylphosphinomethyl)-5-methylcyclopentane, cis-1,2-bis(2-diphosphinomethyl-1,3,5,7-tetramethyl-6,9,10-trioxa-adamantyl)-4,5-dimethylcyclohexane, etc. When used in alkoxycarbonylation and hydroxycarbonylation reactions, this catalyst system can significantly enhance the reaction rates and TON, but the ligand is still used in a relatively high amount.

Patent Literature 9 describes carbonylation ligands and their use in the carbonylation of ethylenically unsaturated compounds. The carbonylation ligand is a bidentate phosphine ligand bridged by a hydrocarbyl aromatic structure having from 5 to 22 cyclic atoms with substituent(s) and at least one 5- or 6-membered aromatic ring. This bidentate phosphine ligand can form a complex with a metal of Group 8, 9 or 10 or a compound thereof. Specific examples of the bidentate phosphine ligand include 1,2-bis(di-t-butyl(phosphinomethyl))-4,5-di(4′-t-butylphenyl)benzene, 1,2-bis(di-t-butyl(phosphinomethyl))-4-t-butylbenzene, 1,2-bis(di-t-butyl(phosphinomethyl))-4,5-di-t-butylbenzene, etc. When used in the carbonylation reactions, the catalyst system containing this bidentate phosphine ligand can produce high TON, but the preparation process of this ligand is very cumbersome and complicated, and some of the ligands show a lower yield and purity.

Patent Literature 10 describes a process for the carbonylation of ethylenically unsaturated compounds, novel carbonylation ligands and catalyst systems incorporating such ligands. The carbonylation ligand used is a bidentate ligand bridged by a hydrocarbyl aromatic structure having at least one aromatic ring. Specific examples of this bidentate ligand include 1-(di-tert-butylphosphinomethyl)-2-(di-o-tolylphosphinomethyl)benzene, 1-(di-tert-pentylphosphinomethyl)-2-(di-o-tolylphosphinomethyl)benzene, etc. The catalyst system containing this bidentate ligand exhibits better stability in the carbonylation reactions, but there still remains room for further improvement in the reaction rate and TON.

The catalyst systems described in the above-mentioned patent applications exhibit high stability and provide relatively high reaction rates in the olefin carbonylation reactions. However, these catalyst systems are still faced with problems, such as a higher proportion of phosphine ligands, longer reaction residence time, rapid catalyst deactivation, and a need of frequent replenishment of new catalysts, which restrict their industrial applications. Therefore, there is a need for improving the existing catalyst systems.

### CITATION LIST

Patent Literature 1: WO1996019434A¹
Patent Literature 2: EP0495547A
Patent Literature 3: U.S. Pat. No. 6,156,934A
Patent Literature 4: CN1429228A
Patent Literature 5: CN1642646A
Patent Literature 6: CN1674990A
Patent Literature 7: CN103223350A
Patent Literature 8: CN101309753A
Patent Literature 9: CN105153241A
Patent Literature 10: CN106854221A

### SUMMARY

### Technical Problem

A primary objective of the present disclosure is to provide a catalyst system suitable for preparation of a carboxylate compound by the carbonylation reaction of an unsaturated compound having a double bond. Furthermore, an objective of the present disclosure is further to provide a method for a carbonylation reaction using the catalyst system.

This catalyst system has a high catalytic activity and requires a smaller amount of bidentate phosphine ligand. Moreover, the product carboxylate compound obtained by the method for the carbonylation reaction of an unsaturated compound having a double bond in the in presence of this catalyst system exhibit higher selectivity and yield; and the conversion rate of the substrate is also higher.

### Solution to Problem

By implementing the following technical solutions, the present disclosure could solve the above-mentioned technical problem.

[1] In the first place, the present disclosure provides a catalyst system for a carbonylation reaction, comprising the following components:
(a) a Group VIII metal or a compound of a Group VIII metal;
(b) a bidentate phosphine ligand; and
(c) an acidic additive;
wherein the bidentate phosphine ligand as component (b) is represented by the following formula (I):

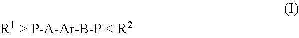

wherein P represents a trivalent phosphorus atom;
Ar represents an aromatic group, A and B each represent a single bond or alkylene, and A and B are at the ortho positions on Ar;
each of R¹ and R², together with the P atom attached thereto, form a monocyclic group that is a phospha monocyclic group having at least 3 carbon atoms on the ring;
the monocyclic group is selected from following groups with or without a substituent: an alicyclic group, a lactone group, a lactam group, a cyclic ketone group, a heterocyclic group containing at least one N atom, and a heterocyclic group containing at least one O atom; the substituent is alkyl, cycloalkyl, substituted or unsubstituted aryl, a halogen atom, a nitrogen-containing group or an oxygen-containing group; and the substituent is attached to the monocyclic group via a single bond or by sharing a plurality of carbon atoms.

[2] The catalyst system according to [1], wherein
A and B each represent C1-C6 alkylene;
Ar represents phenyl or naphthyl;
the monocyclic group is a monocyclic group having 3 to 10 carbon atoms on the ring, and the substituent is alkyl, cycloalkyl, substituted or unsubstituted aryl, a halogen atom, a nitrogen-containing group, or an oxygen-containing group.

[3] The catalyst system according to [1] or [2], wherein
the monocyclic group is selected from following groups with or without a substituent: a phosphacyclopentanyl group, a phosphapropiolactone group, a phosphabutyrolactone group, a phosphavalerolactone group, a phosphacaprolactone group, a phosphaheptanolactone group, a phosphacyclobutanone group, a phosphacyclopentanone group, a phosphacyclohexanone group, a phosphacycloheptanone group, a phosphacyclooctanone group, a phosphatetrahydrofuranyl group, a phosphatetrahydropyrrolyl group, a phosphatetrahydropyranyl group, a phosphahexahydropyridinyl group, a phosphahexahydropyrimidinyl group or a dioxaphosphacyclohexane group, a phosphapropionam group, a phosphabutyrolactam group, a phosphavalerolactam group, a phosphacaprolactam group, or a phosphaheptanolactam group; and
the substituent is alkyl having 1 to 5 carbon atoms, substituted or unsubstituted phenyl, substituted or unsubstituted naphthyl, a chlorine atom, a bromine atom, cyclohexyl, furyl, pyridyl, pyrrolyl, or [2-H]-pyrrolyl.

[4] The catalyst system according to any one of [1] to [3], wherein
the bidentate phosphine ligand is one or more selected from the group consisting of:

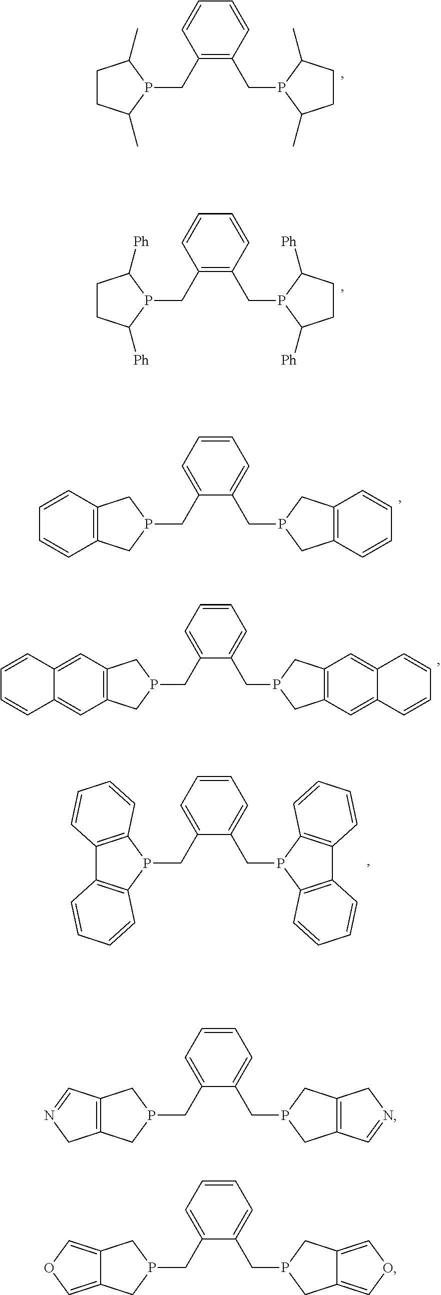

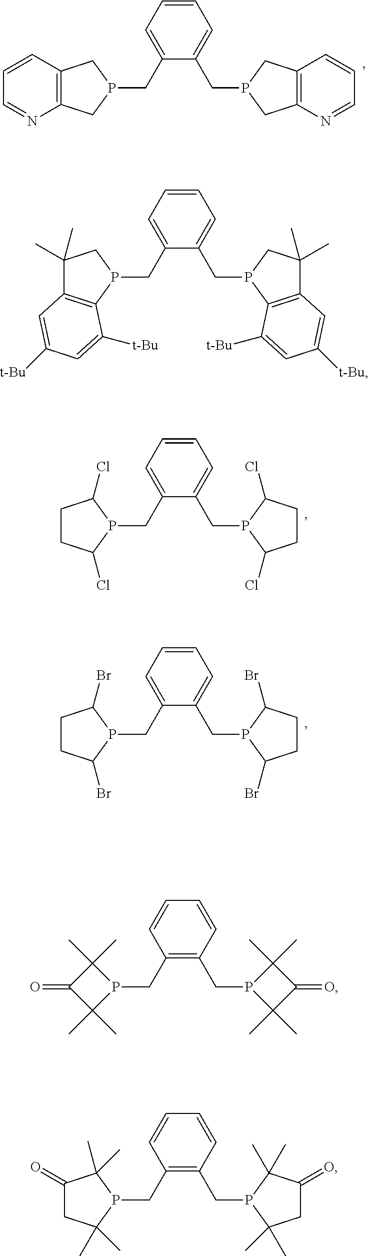 [SMILES: c1ccc(CP2Cc3cccnc3C2)c(CP2Cc3cccnc3C2)c1 ; CC(C)(C)c1cc(C(C)(C)C)c2c(c1)C(C)(C)CP2Cc1ccccc1CP1CC(C)(C)c2cc(C(C)(C)C)cc(C(C)(C)C)c21 ; ClC1CCC(Cl)P1Cc1ccccc1CP1C(Cl)CCC1Cl ; BrC1CCC(Br)P1Cc1ccccc1CP1C(Br)CCC1Br ; CC1(C)C(=O)C(C)(C)P1Cc1ccccc1CP1C(C)(C)C(=O)C1(C)C ; CC1(C)CC(=O)C(C)(C)P1Cc1ccccc1CP1C(C)(C)CC(=O)C1(C)C]

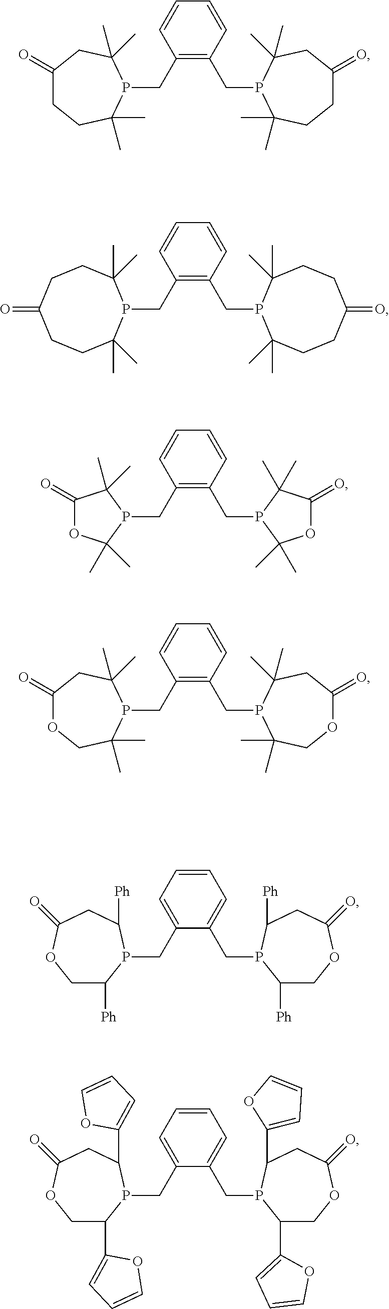 [SMILES: CC1(C)CCC(=O)CC(C)(C)P1Cc1ccccc1CP1C(C)(C)CCC(=O)CC1(C)C ; CC1(C)CCC(=O)CCC(C)(C)P1Cc1ccccc1CP1C(C)(C)CCC(=O)CCC1(C)C ; CC1(C)OC(=O)C(C)(C)P1Cc1ccccc1CP1C(C)(C)OC(=O)C1(C)C ; CC1(C)COC(=O)CC(C)(C)P1Cc1ccccc1CP1C(C)(C)COC(=O)CC1(C)C ; O=C1CC(c2ccccc2)P(Cc2ccccc2CP2C(c3ccccc3)COC(=O)CC2c2ccccc2)C(c2ccccc2)CO1 ; O=C1CC(c2ccco2)P(Cc2ccccc2CP2C(c3ccco3)COC(=O)CC2c2ccco2)C(c2ccco2)CO1]

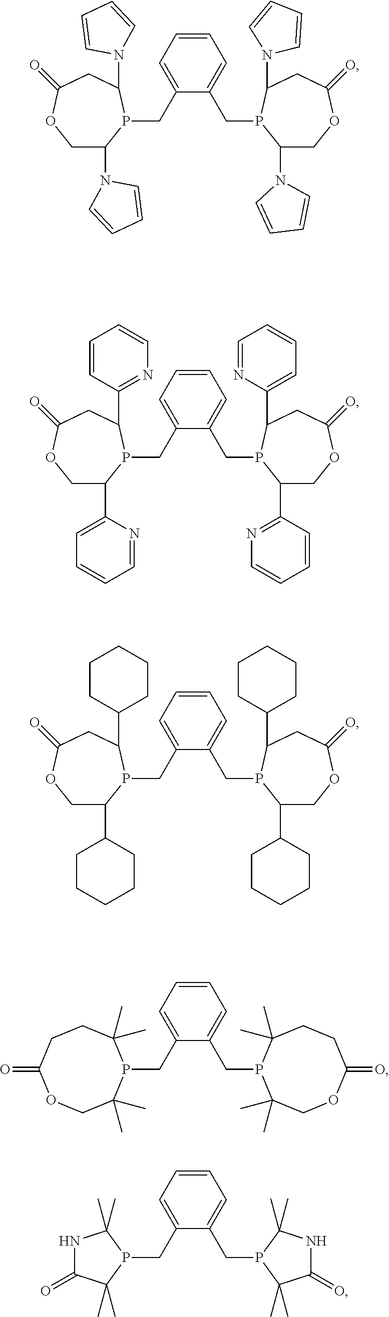 [SMILES: O=C1CC(n2cccc2)P(Cc2ccccc2CP2C(n3cccc3)COC(=O)CC2n2cccc2)C(n2cccc2)CO1 ; O=C1CC(c2ccccn2)P(Cc2ccccc2CP2C(c3ccccn3)COC(=O)CC2c2ccccn2)C(c2ccccn2)CO1 ; O=C1CC(C2CCCCC2)P(Cc2ccccc2CP2C(C3CCCCC3)COC(=O)CC2C2CCCCC2)C(C2CCCCC2)CO1 ; CC1(C)CCC(=O)OCC(C)(C)P1Cc1ccccc1CP1C(C)(C)CCC(=O)OCC1(C)C ; CC1(C)NC(=O)C(C)(C)P1Cc1ccccc1CP1C(C)(C)NC(=O)C1(C)C]

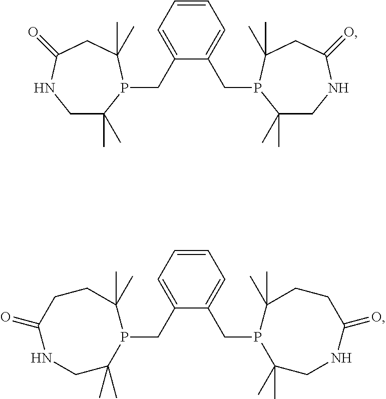 [SMILES: CC1(C)CNC(=O)CC(C)(C)P1Cc1ccccc1CP1C(C)(C)CNC(=O)CC1(C)C ; CC1(C)CCC(=O)NCC(C)(C)P1Cc1ccccc1CP1C(C)(C)CCC(=O)NCC1(C)C]

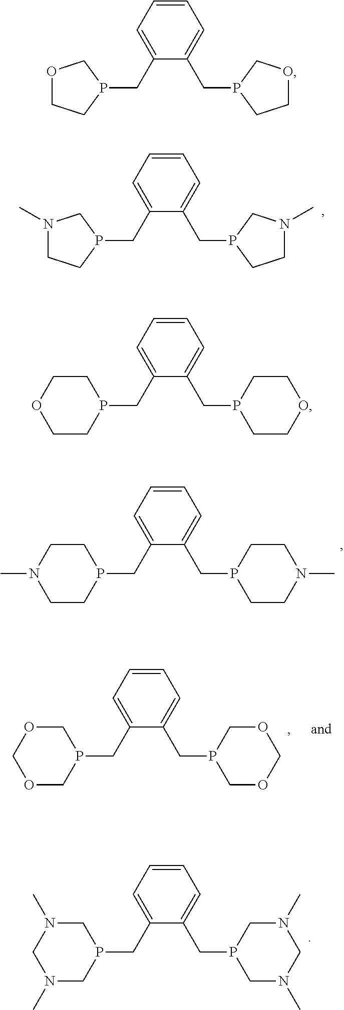

[5] The catalyst system according to any one of [1] to [4], wherein a molar ratio of the component (b) to the component (a) is 2:1 to 10:1, preferably 2:1 to 5:1;
a molar ratio of the component (c) to the component (a) is 2:1 to 100:1, preferably 10:1 to 50:1.

[6] The catalyst system according to any one of [1] to [5], wherein
in the component (a):
the Group VIII metal comprises cobalt, nickel, palladium, rhodium, ruthenium, iridium or platinum;
the compound of the Group VIII metal comprises: a compound formed of the Group VIII metal and the following substance: sulfuric acid, nitric acid, phosphoric acid, formic acid, acetic acid, propionic acid, trichloroacetic acid, trifluoroacetic acid, methanesulfonic acid, chlorosulfonic acid, fluorosulfonic acid, trifluoromethanesulfonic acid, benzenesulfonic acid, naphthalenesulfonic acid, toluenesulfonic acid, sulfonated ion exchange resin, or perhalogenic acid; or a complex of zero-valent palladium, rhodium, iridium, platinum, or ruthenium;
the acidic additive as the component (c) is an acid having a pKa value of less than 5, preferably less than 4, more preferably less than 3 in an aqueous solution at 25° C.

[7] The catalyst system according to any one of [1] to [6], wherein
the acidic additive as the component (c) comprises at least one of methanesulfonic acid, trifluoromethanesulfonic acid, tert-butylsulfonic acid, p-toluenesulfonic acid, 2-hydroxypropyl-2-sulfonic acid, 2,4,6-trimethylmethanesulfonic acid, perchloric acid, phosphoric acid, methylphosphoric acid, and sulfuric acid.

[8] Furthermore, the present disclosure further provides a method for a carbonylation reaction, the method comprising reacting an unsaturated compound having a double bond with carbon monoxide and an alcohol in the presence of a catalyst system;
wherein the catalyst system is the catalyst system according to any one of [1] to [7].

[9] The method according to [8], wherein a molar ratio of the unsaturated compound having a double bond to the carbon monoxide is 1:1 to 100:1, preferably 2:1 to 50:1;
a molar ratio of the unsaturated compound having a double bond to the component (a) in the catalyst system is 50:1 to 600:1, preferably 100:1 to 300:1;
a mass ratio of the alcohol to the component (a) in the catalyst system is 500:1 to 20000:1, preferably 5000:1 to 15000:1.

[10] The method according to [8] or [9], wherein the reaction is conducted under the following operating conditions:
a reaction pressure is at 1 to 20 MPa, preferably 1 to 10 MPa; and a reaction temperature is at 50 to 200° C., preferably 60 to 150° C.

[11] The method according to any one of [8] to [10], wherein
the unsaturated compound having a double bond is a substituted or unsubstituted C2-C20 olefin, preferably a substituted or unsubstituted C2-C16 olefin; when the olefin has a substituent, the substituent is C1-C10 alkyl, C6-C12 aryl, C1-C4 alkoxy, halogen-substituted C6-C12 aryl, a C2-C6 ester group, or a nitrogen-containing heterocyclic group; the olefin is preferably a C2-C6 olefin;
the alcohol is a substituted or unsubstituted linear or branched C1-C10 alkanol; when the alcohol is an alcohol having a substituent, the substituent is C1-C6 alkyl, C6-C20 aryl, C2-C10 heterocyclyl, halogen, cyano or nitro, preferably C1-C6 alkyl or C6-C10 aryl; the alcohol is preferably a C1-C6 monohydric alkanol.

### Advantageous Effects of the Invention

Since the catalyst system provided herein contains a specific bidentate phosphine ligand, when used in the olefin carbonylation reactions, it exhibits a higher catalytic activity and significantly reduces the amounts of the bidentate phosphine ligand and the Group VIII metal or the compound thereof in the catalyst system.

Moreover, the method for the carbonylation reaction of an unsaturated compound having a double bond using the catalyst system according to the present disclosure can achieve excellent technical effects such as good product selectivity, a high product yield, a high conversion rate of the substrate, and a long service life of the catalyst system.

Furthermore, the catalyst system of the present disclosure could effectively catalyze the olefin carbonylation reaction to synthesize carboxylate products, so it can improve the catalytic efficiency of the reaction and reduce the residence time of the substrate.

### BRIEF DESCRIPTION OF THE DRAWINGS

FIG. 1 shows the free energy potential energy surface for the olefin carbonylation reaction catalyzed by a palladium catalyst in a strong acid system (where the bidentate phosphine ligand is represented by, [symbol] olefin is exemplified by ethylene, the acid is exemplified by methanesulfonic acid, and the alcohol is exemplified by methanol).

### DETAILED DESCRIPTION

The following is a detailed description of the present disclosure. The technical features described below will be described based on the representative embodiments and specific examples of the present disclosure, but the present disclosure is not limited thereto. The following should be noted:

The numerical range represented by “numerical value A to numerical value B” used in the present specification refers to the range including the endpoint values A and B.

In the present specification, the term “basically” or “substantially” means that the standard deviation from the theoretical model or theoretical data is within a range of 5%, preferably 3%, more preferably 1%.

In the present specification, the term “may” involves both the meaning of doing something and the meaning of not doing something.

The term “monocyclic group” used in the present specification means that the phosphorus atom is positioned in only one ring structure, rather than on multiple rings simultaneously.

In the present disclosure, the term “optional” or “optionally” means that the event or case described subsequently may or may not occur, and the description includes the case where the event occurs and the case where the event does not occur.

Phrases such as “some specific/preferred embodiments”, “other specific/preferred embodiments”, “embodiments” and the like referred to in the present specification mean that particular elements (for example, features, structures, properties and/or characteristics) described in relation to this embodiment are included in at least one of the embodiments described herein, and may or may not exist in other embodiments. Additionally, it should be appreciated that the elements may be combined in any suitable manner into various embodiments.

The present disclosure provides a catalyst system useful for the carbonylation reaction of an unsaturated compound having a double bond. Since this catalyst system contains a specific bidentate phosphine ligand, when used in olefin carbonylation reactions, it exhibits a higher catalytic activity and significantly reduces the amounts of the bidentate phosphine ligand and the Group VIII metal or a compound thereof in the catalyst system.

### (Bidentate Phosphine Ligand)

In the present disclosure, the bidentate phosphine ligand has a structure represented by the following formula (I):

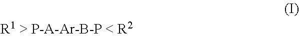

wherein P represents a trivalent phosphorus atom.
Ar represents an aromatic group, which may or may not have an alkyl substituent. In some specific embodiments of the present disclosure, Ar may be phenyl or naphthyl, preferably phenyl.
A and B are the same or different and may each independently represent a single bond or alkylene, and A and B are located at the ortho positions on the Ar group as an aromatic group. In some preferred embodiments, A or B may be C1-C6 alkylene, typically methylene, ethylene, propylene, butylene, pentylene or hexylene, for example.

Further, each of R¹ and R², together with the P atom attached thereto, form a monocyclic group that is a phospha monocyclic group having at least 3 carbon atoms on the ring; preferably, the monocyclic group is a monocyclic group having 3 to 10 carbon atoms on the ring.

In the present disclosure, the aforesaid monocyclic group is selected from following groups with or without a substituent: an alicyclic group, a lactone group, a lactam group, a cyclic ketone group, a heterocyclic group containing at least one N atom, or a heterocyclic group containing at least one O atom, preferably a lactone group and a lactam group, more preferably a lactone group.

Furthermore, the monocyclic group is preferably a phosphacyclopentanyl group, a phosphapropiolactone group, a phosphabutyrolactone group, a phosphavalerolactone group, a phosphacaprolactone group, a phosphaheptanolactone group, a phosphacyclobutanone group, a phosphacyclopentanone group, a phosphacyclohexanone group, a phosphacycloheptanone group, a phosphacyclooctanone group, a phosphatetrahydrofuranyl group, a phosphatetrahydropyrrolyl group, a phosphatetrahydropyranyl group, a phosphahexahydropyridinyl group, a phosphahexahydropyrimidinyl group, a dioxaphosphacyclohexane group, a phosphapropionam group, a phosphabutyrolactam group, a phosphavalerolactam group, a phosphacaprolactam group, or a phosphaheptanolactam group; preferably a phosphapropiolactone group, a phosphabutyrolactone group, a phosphavalerolactone group, a phosphacaprolactone group, a phosphaheptanolactone group, a phosphapropionam group, a phosphabutyrolactam group, a phosphavalerolactam group, a phosphacaprolactam group, or a phosphaheptanolactam group; more preferably a phosphapropiolactone group, a phosphabutyrolactone group, a phosphavalerolactone group, a phosphacaprolactone group, or a phosphaheptanolactone group.

The substituent of the monocyclic group is alkyl, cycloalkyl, substituted or unsubstituted aryl, a halogen atom, a nitrogen-containing group, or an oxygen-containing group, and the substituent may be attached to the monocyclic group via a single bond or by sharing a plurality of carbon atoms.

The substituent of the monocyclic group may be one or more. In some preferred embodiments of the present disclosure, the alkyl may be C1-C10 alkyl; the cycloalkyl may be cyclopropyl, cyclobutyl, cyclopentyl, cyclohexyl or cycloheptyl; the substituted or unsubstituted aryl may be substituted or unsubstituted phenyl or substituted or unsubstituted naphthyl; the halogen atom may be a fluorine, chlorine, bromine or iodine atom; the nitrogen-containing group may be pyridyl, pyrrolyl or [2-H]-pyrrolyl; the oxygen-containing group may be furanyl.

In further preferred embodiments, the substituent of the monocyclic group is preferably alkyl having 1 to 5 carbon atoms, substituted or unsubstituted phenyl, substituted or unsubstituted naphthyl, a chlorine atom, a bromine atom, cyclohexyl, furyl, pyridyl, pyrrolyl, or [2-H]-pyrrolyl.

More specifically, specific examples of the bidentate phosphine ligand of the present disclosure may include, but are not limited to, the following ligands:

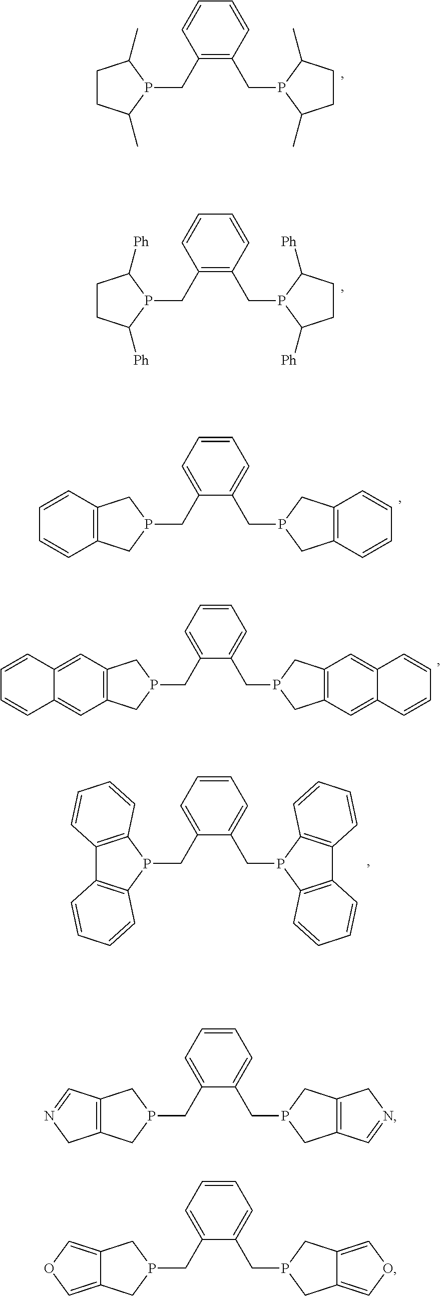

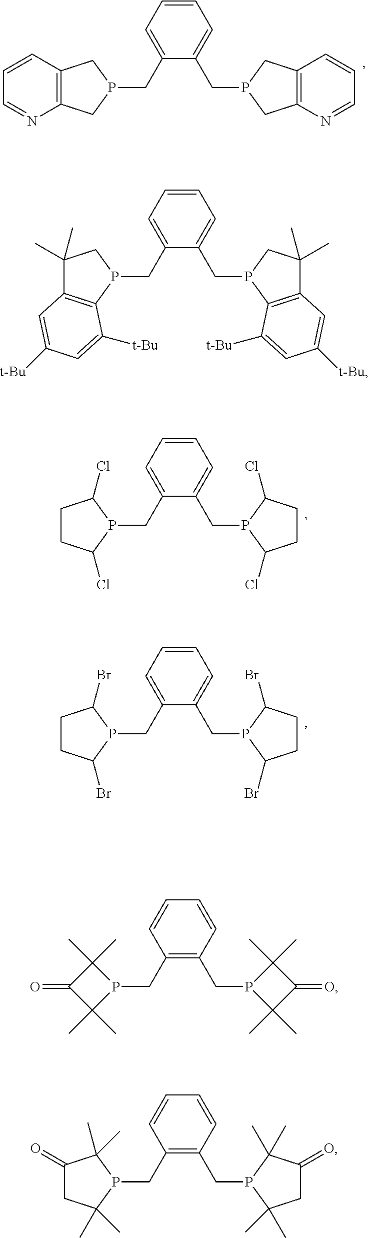 [SMILES: c1ccc(CP2Cc3cccnc3C2)c(CP2Cc3cccnc3C2)c1 ; CC(C)(C)c1cc(C(C)(C)C)c2c(c1)C(C)(C)CP2Cc1ccccc1CP1CC(C)(C)c2cc(C(C)(C)C)cc(C(C)(C)C)c21 ; ClC1CCC(Cl)P1Cc1ccccc1CP1C(Cl)CCC1Cl ; BrC1CCC(Br)P1Cc1ccccc1CP1C(Br)CCC1Br ; CC1(C)C(=O)C(C)(C)P1Cc1ccccc1CP1C(C)(C)C(=O)C1(C)C ; CC1(C)CC(=O)C(C)(C)P1Cc1ccccc1CP1C(C)(C)CC(=O)C1(C)C]

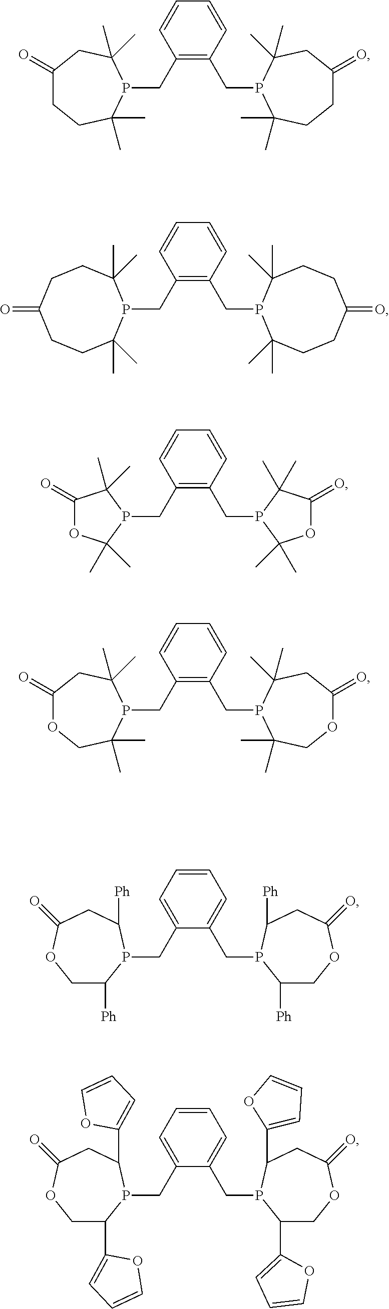 [SMILES: CC1(C)CCC(=O)CC(C)(C)P1Cc1ccccc1CP1C(C)(C)CCC(=O)CC1(C)C ; CC1(C)CCC(=O)CCC(C)(C)P1Cc1ccccc1CP1C(C)(C)CCC(=O)CCC1(C)C ; CC1(C)OC(=O)C(C)(C)P1Cc1ccccc1CP1C(C)(C)OC(=O)C1(C)C ; CC1(C)COC(=O)CC(C)(C)P1Cc1ccccc1CP1C(C)(C)COC(=O)CC1(C)C ; O=C1CC(c2ccccc2)P(Cc2ccccc2CP2C(c3ccccc3)COC(=O)CC2c2ccccc2)C(c2ccccc2)CO1 ; O=C1CC(c2ccco2)P(Cc2ccccc2CP2C(c3ccco3)COC(=O)CC2c2ccco2)C(c2ccco2)CO1]

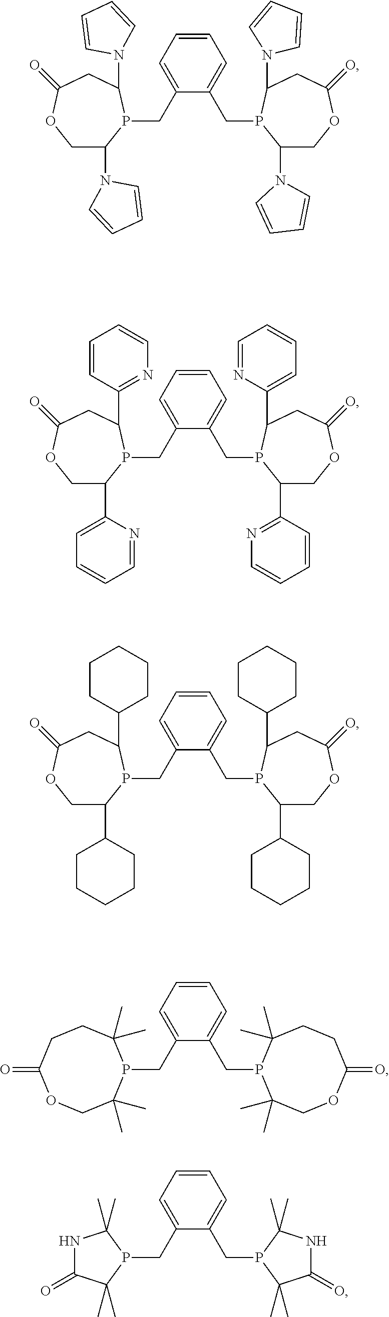 [SMILES: O=C1CC(n2cccc2)P(Cc2ccccc2CP2C(n3cccc3)COC(=O)CC2n2cccc2)C(n2cccc2)CO1 ; O=C1CC(c2ccccn2)P(Cc2ccccc2CP2C(c3ccccn3)COC(=O)CC2c2ccccn2)C(c2ccccn2)CO1 ; O=C1CC(C2CCCCC2)P(Cc2ccccc2CP2C(C3CCCCC3)COC(=O)CC2C2CCCCC2)C(C2CCCCC2)CO1 ; CC1(C)CCC(=O)OCC(C)(C)P1Cc1ccccc1CP1C(C)(C)CCC(=O)OCC1(C)C ; CC1(C)NC(=O)C(C)(C)P1Cc1ccccc1CP1C(C)(C)NC(=O)C1(C)C]

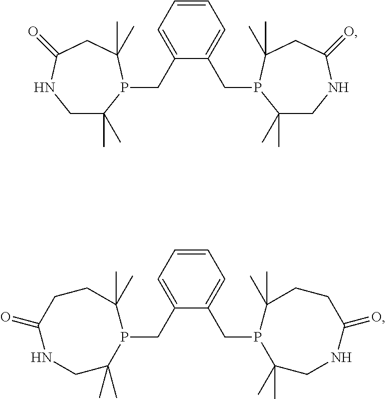 [SMILES: CC1(C)CNC(=O)CC(C)(C)P1Cc1ccccc1CP1C(C)(C)CNC(=O)CC1(C)C ; CC1(C)CCC(=O)NCC(C)(C)P1Cc1ccccc1CP1C(C)(C)CCC(=O)NCC1(C)C]

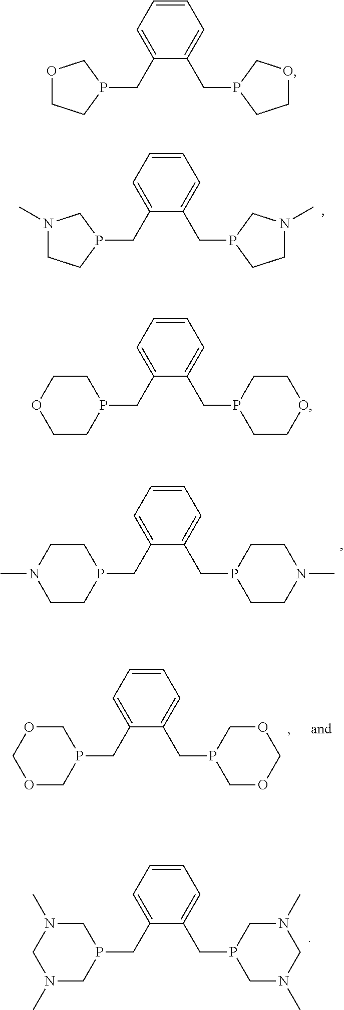

Due to the presence of the cyclic structure such as a phosphamonocyclic alicyclic group, a phosphamonocyclic lactone group, a phosphamonocyclic cyclic ketone group, a phosphamonocyclic lactam group, a phosphamonocyclic N-containing group or a phosphamonocyclic O-containing group of the phosphine ligand, the bidentate phosphine ligand of the present disclosure is able to cause compression of the C—P—C bond angle, such that the bidentate phosphine ligand having such a structure could be stably complexed with a Group VIII metal to form a square-like planar geometry with a suitable bite angle, and the structure is not easily destroyed.

At the same time, the bidentate phosphine ligand is an extremely weak base, resulting in a low ability to provide electron density to the metal. This leads to a higher reactivity of the electrophilic metal center, and may accelerate the rate-determining methanol decomposition step in the olefin carbonylation reaction. Therefore, in the catalyst system containing this bidentate phosphine ligand, the complex of the Group VIII metal and the bidentate phosphine ligand is very stable and allows the catalyst system to show a high catalytic activity and accelerate the carbonylation reaction.

The synthetic method for the bidentate phosphine ligand provided herein is not particularly limited in principle. In some preferred embodiments of the present disclosure, the bidentate phosphine ligand may be obtained by subjecting a phosphorus-containing cyclic compound to the boronation reaction and then reacting with a halogenated aromatic compound.

For the boronation reaction, it is possible to react borane with a phosphorus-containing cyclic compound to obtain, for example, a phosphine (cycloalkane/cyclic ester/cyclic ketone)-borane complex. Borane may preferably be BH₃. In addition, the source of the phosphorus-containing cyclic compound is not particularly limited in the present disclosure and it may be obtained by a common synthetic method in the art or may be commercially available.

### (Catalyst System)

The catalyst system provided herein comprises the following components:
(a) a Group VIII metal or a compound of a Group VIII metal;
(b) a bidentate phosphine ligand; and
(c) an acidic additive;
wherein the bidentate phosphine ligand as the component (b) is the bidentate phosphine ligand as described above.

In some specific embodiments, in the catalyst system provided herein, the molar ratio of the component (b) to the component (a) is 2:1 to 10:1, preferably 2:1 to 5:1. When the molar ratio of the component (b) to the component (a) is in the above range, the catalyst system can achieve the technical effects of good stability and a high catalytic activity.

In some specific embodiments, in the catalyst system provided herein, the molar ratio of the component (c) to the component (a) is 2:1 to 100:1, preferably 10:1 to 50:1. When the molar ratio of the component (c) to the component (a) is in the above range, it is possible to achieve the technical effects of a high reaction conversion rate and selectivity.

Additionally, in the catalyst system provided herein, the Group VIII metal in the component (a) includes one or more of cobalt, nickel, palladium, rhodium, ruthenium, iridium or platinum.

The compound of the Group VIII metal includes one or more of the following compounds: a compound formed of the Group VIII metal and the following substance: sulfuric acid, nitric acid, phosphoric acid, formic acid, acetic acid, propionic acid, trichloroacetic acid, trifluoroacetic acid, methanesulfonic acid, chlorosulfonic acid, fluorosulfonic acid, trifluoromethanesulfonic acid, benzenesulfonic acid, naphthalenesulfonic acid, toluenesulfonic acid, sulfonated ion exchange resin, or perhalogenic acid; or a complex of zero-valent palladium, rhodium, iridium, platinum, or ruthenium.

In the catalyst system provided herein, the acidic additive as the component (c) is an acid having a pKa value of less than 5, preferably less than 4, more preferably less than 3 in an aqueous solution at 25° C.

In some preferred embodiments of the present disclosure, the acidic additive as the component (c) comprises at least one of methanesulfonic acid, trifluoromethanesulfonic acid, tert-butylsulfonic acid, p-toluenesulfonic acid, 2-hydroxypropyl-2-sulfonic acid, 2,4,6-trimethylmethanesulfonic acid, perchloric acid, phosphoric acid, methylphosphoric acid, and sulfuric acid.

The catalytic process of the above-mentioned catalyst system provided herein can be described as follows:

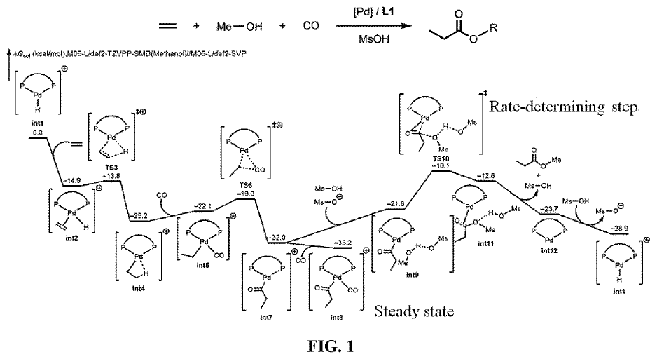

Referring to FIG. 1, based on the existing example of the carbonylation reaction of an unsaturated compound having a double bond (taking ethylene as an example for illustration herein below), the present disclosure elucidates the microscopic process of the olefin carbonylation reaction catalyzed by a palladium catalyst in a strong acid system based on the density functional theory calculation, and establishes the free energy potential energy surface change throughout the catalytic cycle.

The reaction starts with the active catalyst—a protonated palladium species int1. Firstly, it undergoes the ethylene coordination (int2) and a rapid insertion process (TS3, free energy barrier of 1.1 kcal/mol) to yield an intermediate int4 with an agostic hydrogen bond. Subsequently, the coordination of carbon monoxide (int5) and a rapid migratory insertion process of a carbonyl group (TS3, free energy barrier of 3.1 kcal/mol) occur to generate the tri-coordinated intermediate int7. At this moment, carbon monoxide can undergo additional coordination with int7 to generate a more stable tetra-coordinated steady-state intermediate int8. Afterwards, alcohols such as methanol participate in the nucleophilic attack on the carbonyl group (TS10, free energy barrier of 23.1 kcal/mol) with the help of acid anions to generate int11. This challenging carbon-oxygen bond formation process is the rate-determining step of the entire catalytic cycle. Finally, the generated zero-valent palladium species int12 reacts with the strong acid species in the system to regenerate the active catalyst int1.

Based on theoretical calculations of the catalytic process, the present disclosure clarifies, at a microscopic level, that in the ethylene carbonylation reaction occurring in a palladium-catalyzed strong acid system, carbon monoxide has a certain toxic effect on the reaction, while anions capable of acting as a Bronsted base in an acidic medium are required to exist in the system. The large-steric bidentate ligand skeleton involved in the present disclosure is effective in inhibiting the toxic effect of carbon monoxide, such that the catalyst system has a high catalytic activity.

### (Carbonylation Reaction)

The method for the carbonylation reaction according to the present disclosure, the method comprises: reacting an unsaturated compound having a double bond with carbon monoxide and an alcohol in the presence of a catalyst system; wherein the catalyst system is the catalyst system according to the present disclosure.

In the method for the carbonylation reaction according to the present disclosure, in preferred cases, the molar ratio of the unsaturated compound having a double bond to carbon monoxide is 1:1 to 100:1, preferably 2:1 to 50:1; the molar ratio of the unsaturated compound having a double bond to the component (a) in the catalyst system is 50:1 to 600:1, preferably 100:1 to 300:1; and the mass ratio of the alcohol to the component (a) in the catalyst system is 500:1 to 20000:1, preferably 5000:1 to 15000:1.

In the method for the carbonylation reaction according to the present disclosure, in preferred cases, the reaction is conducted under the following operating conditions: the reaction pressure is at 1 to 20 MPa, preferably 1 to 10 MPa; and the reaction temperature is at 50 to 200° C., preferably 60 to 150° C.

Furthermore, the unsaturated compound having a double bond in the present disclosure may typically be a substituted or unsubstituted olefin. In some specific embodiments of the present disclosure, such a compound may be a substituted or unsubstituted C2-C20 olefin, preferably a substituted or unsubstituted C2-C16 olefin. When the olefin has a substituent, the substituent may be C1-C10 alkyl, C6-C12 aryl, C1-C4 alkoxy, halogen-substituted C6-C12 aryl, a C2-C6 ester group, or a nitrogen-containing heterocyclic group.

In further preferred embodiments of the present disclosure, the unsaturated compound having a double bond is a C2-C6 olefin.

Additionally, the aforesaid unsaturated compound having a double bond may be used alone or in a mixture of two or more, but it is preferable to use only one.

The alcohol in the aforesaid carbonylation reaction is a substituted or unsubstituted linear or branched C1-C10 alkanol; when the alcohol is an alcohol having a substituent, the substituent is C1-C6 alkyl, C6-C20 aryl, C2-C10 heterocyclyl, halogen, cyano or nitro; preferably, the substituent is C1-C6 alkyl or C6-C10 aryl.

In further preferred embodiments of the present disclosure, the alcohol is a C1-C6 monohydric alkanol.

Additionally, the aforesaid alcohol may be used alone or in a mixture of two or more, but it is preferable to use only one.

According to the method for the carbonylation reaction provided herein, more preferably, the method for the carbonylation reaction using ethylene as an unsaturated compound having a double bond is as follows:

Methanol, a compound of a Group VIII metal, a bidentate phosphine ligand, and an acidic additive are added in certain amounts to an autoclave, and the autoclave is sealed. Subsequently, a gas mixture of ethylene and carbon monoxide at a certain ratio is introduced into the autoclave while stirring, and then the pressure is gradually increased to the reaction pressure, while heating the autoclave to the reaction temperature. After reacting for a period of time, samples are taken for GC chromatographic analysis.

The compound of a Group VIII metal may be any one of the compounds as listed above. The acidic additive is preferably methanesulfonic acid, benzenesulfonic acid or p-toluenesulfonic acid.

The method for the carbonylation reaction of an unsaturated compound having a double bond conducted in the presence of the catalyst system provided herein can achieve the technical effects of good product selectivity, a high product yield, a high conversion rate of the substrate, and a long service life of the catalyst system.

### EXAMPLES

The present disclosure is further described below with reference to specific examples and comparative examples.

### Preparation Example 1

### Preparation of Bidentate Phosphine Ligand 1

### (1) Preparation of 2,5-dimethylphosphane-borane complex (1-a)

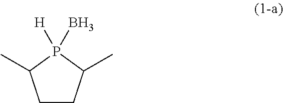

To a solution of 6.0 g (24 mmol) of P (SiMe₃)₃ in 300 mL of THF, 25.1 mmol (1.05 equiv/P) of MeLi (15.7 mL, 1.6 M ether solution) was added. The solution was stirred for 12 h and the solvent was removed under vacuum. The resulting white solid was dissolved into 300 mL of ether to yield a yellow solution. 4.3 g (24 mmol) of 3,6-hexanediol cyclic sulfate was slowly added to the yellow solution, and the yellow disappeared. After the solution was stirred for 2 h, 20 mL of methanol was slowly added. The reaction mixture was stirred for 24 h and the solvent was carefully removed under vacuum at room temperature to afford a white (sometimes yellow) solid. (Note: it was difficult to completely remove methanol from this material and excess MeLi was required to be used in the next step.) The solid was dissolved into 300 mL of THF and 25.1 mmol of MeLi (15.7 mL, 1.6 M ether solution) was added. The resulting mixture was stirred for 2 h, and 1.5 to 2 equiv of additional MeLi was added to completely convert the mixture of secondary phosphine and primary phosphine sulfonate as an intermediate. Next, 48 mmol (2 equiv/P) of BH₃-THF solution (48 mL, 1M THF solution) was added. The resulting residue was extracted with dichloromethane (200 mL). The resulting solution was concentrated under vacuum. The residue was extracted with hexane (200 mL) and then filtered through a silica gel pad. The solvent was removed under vacuum to afford 2,5-dimethylphosphane-borane complex (1-a) as a colorless oily product (1.9 g, 61% yield).

### (2) Preparation of bis(phosphane-borane) (1-b)

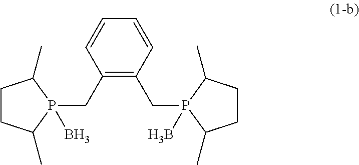

To an ice-cooled mixture of 2,5-dimethylphosphane-borane complex (1-a) (1.9 g, 14.6 mmol) and Bu₄NBr (158 mg, 0.5 mmol) in 30% aqueous potassium hydroxide solution (100 mL) and toluene (30 mL), an appropriate amount of o-dibenzyl bromide (1.73 g, 6.6 mmol, 1.1 equiv P/Br) was added, and the mixture was stirred vigorously at room temperature for 16 h. Thereafter, ether (100 mL) was added and the organic phases were collected. The organic phases were washed with water (3×10 mL) and saline (1×10 mL) and dried over anhydrous MgSO₄. After evaporation of the solvent under vacuum, the crude product was purified by crystallization from hexane to afford bis(phosphane-borane) (1-b) as a white solid (1.6 g, 67% yield).

### (3) Preparation of Bidentate Phosphine Ligand 1

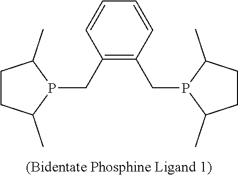 [SMILES: CC1CCC(C)P1Cc1ccccc1CP1C(C)CCC1C]

To a 50-mL Schlenk flask, bis(phosphane-borane) (1-b) (362 mg, 1.0 mmol), DABCO (1,4-diazabicyclo[2,2,2]octane, 247 mg, 2.2 mmol), and toluene (15 mL) were charged, and the mixture was stirred at 50° C. for 15 h. The mixture was cooled at room temperature, filtered through a silica pad, and eluted with 150 mL of degassed hexane/ether (10/1). The filtrate was concentrated under vacuum to afford bidentate phosphine ligand 1 as a colorless oily product (300 mg, 90%).

NMR data of bidentate phosphine ligand 1:

¹H NMR (400 MHz, CDCl₃): δ=7.18-7.09 (m, 4H), 3.62 (dd, J=6.9, 14.7 Hz, 2H), 2.77 (pseudo t, J=15.2 Hz, 2H), 2.26-2.06 (m, 8H), 1.49-1.34 (m, 4H), 1.32 (dd, J=6.4, 13.0 Hz, 6H), 0.86 (dd, J=6.9, 15.7 Hz, 6H).

¹³C NMR (100 MHz, CDCl₃): δ=132.82, 130.58, 127.06, 34.70, 34.44, 34.34, 32.05, 27.17, 15.11, 13.69.

³¹P NMR (162 MHz, CDCl₃): δ=38.68.

### Preparation Example 2

### Preparation of Bidentate Phosphine Ligand 2

### (1) Preparation of 2,5-diphenylphosphane-borane Complex (2-a)

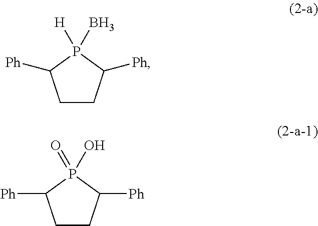

At room temperature and under nitrogen atmosphere, phenylsilane (1.68 g, 15.5 mmol) was added dropwise via a syringe to a suspension of 1-hydroxy-1-oxo-2,5-diphenylphosphine (2-a-1) (3.50 g, 12.0 mmol) in toluene (20 mL) while stirring. Once the dropwise addition was completed, the reaction solution was heated at 110° C. The mixture was reacted at 110° C. for 16 h, cooled at room temperature, and then concentrated under vacuum to afford an opaque gel. Under nitrogen atmosphere, the gel was placed in the degassed THF (20 mL) and the solution was cooled at 0° C. Borane-methyl sulfide complex (4.88 mL, 51.4 mmol) was added dropwise via a syringe. Once the addition was completed, the reaction solution was heated at room temperature. After reacting at room temperature for 16 h, the reaction solution was concentrated under vacuum to afford a white solid. The solid was purified by flash column chromatography (heptane:ethyl acetate=9:1) to afford 2,5-diphenylphosphane-borane complex (2-a) as a white solid product (3.12 g, 95% yield).

### (2) Preparation of bis(2,5-diphenylphosphane-borane) (2-b)

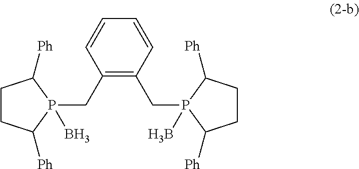

To an ice-cooled mixture of 2,5-diphenylphosphane-borane complex (2-a) (3.7 g, 14.6 mmol) and Bu₄NBr (158 mg, 0.5 mmol) in 30% aqueous potassium hydroxide solution (100 mL) and toluene (30 mL), an appropriate amount of o-dibenzyl bromide (1.73 g, 6.6 mmol, 1.1 equiv P/Br) was added, and the mixture was stirred vigorously at room temperature for 16 h. Thereafter, ether (100 mL) was added and the organic phases were collected. The organic phases were washed with water (3×10 mL) and saline (1×10 mL) and dried over anhydrous MgSO₄. After evaporation of the solvent under vacuum, the crude product was purified by crystallization from hexane to afford bis(2,5-diphenylphosphane-borane) (2-b) as a white solid (2.4 g, 60% yield).

### (3) Preparation of Bidentate Phosphine Ligand 2

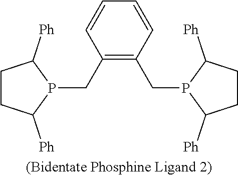 [SMILES: c1ccc(C2CCC(c3ccccc3)P2Cc2ccccc2CP2C(c3ccccc3)CCC2c2ccccc2)cc1]

To a 50-mL Schlenk flask, bis(2,5-diphenylphosphane-borane) (2-b) (610 mg, 1.0 mmol), DABCO (1,4-diazabicyclo[2,2,2]octane, 247 mg, 2.2 mmol), and toluene (15 mL) were charged and the mixture was stirred at 50° C. for 15 h. The mixture was cooled at room temperature, filtered through a silica pad, and eluted with 150 mL of degassed hexane/ether (10/1). The filtrate was concentrated under vacuum to afford bidentate phosphine ligand 2 as a colorless oily product (495 mg, 85%).

NMR data of bidentate phosphine ligand 2:

¹H NMR (400 MHz, CDCl₃): δ=7.37-7.29 (m, 4H), 7.26-7.17 (m, 2H), 7.15-7.06 (m, 5H), 7.04-6.95 (m, 1H), 3.20 (ddd, J=13.7, 1.8, 1.1 Hz, 2H), 3.04 (tt, J=5.0, 1.0 Hz, 2H), 2.16-2.06 (m, 2H), 2.06-1.99 (m, 2H).

¹³C NMR (100 MHz, CDCl₃): δ=138.61, 138.59, 130.08, 129.45, 127.94, 126.62, 126.41, 52.74, 33.04, 31.31.

³¹P NMR (162 MHz, CDCl₃): δ=54.36.

### Preparation Example 3

### Preparation of Bidentate Phosphine Ligand 3

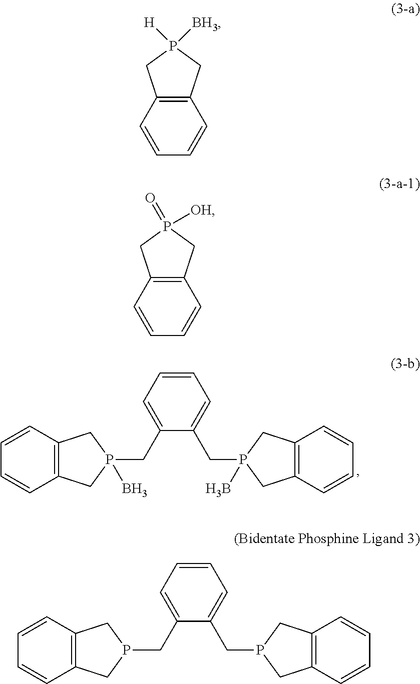

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (3-a-1), replacing 2,5-diphenylphosphane-borane complex (2-a) with an equimolar amount of a compound of formula (3-a), and replacing bis(2,5-diphenylphosphane-borane) (2-b) with an equimolar amount of a compound of formula (3-b), to finally afford bidentate phosphine ligand 3 as a colorless oily product (329 mg, 88%).

NMR data of bidentate phosphine ligand 3:

¹H NMR (400 MHz, CDCl₃): δ=7.20-7.00 (m, 3H), 3.14 (ddd, J=13.8, 12.9, 1.1 Hz, 1H), 3.07 (ddd, J=13.7, 12.8, 0.9 Hz, 1H), 2.95 (dd, J=13.7, 1.0 Hz, 1H).

¹³C NMR (100 MHz, CDCl₃): δ=137.78, 137.22, 130.05, 126.49, 126.46, 126.40, 126.33, 126.26, 126.23, 40.84, 32.51.

³¹P NMR (162 MHz, CDCl₃): δ=40.79.

### Preparation Example 4

### Preparation of Bidentate Phosphine Ligand 4

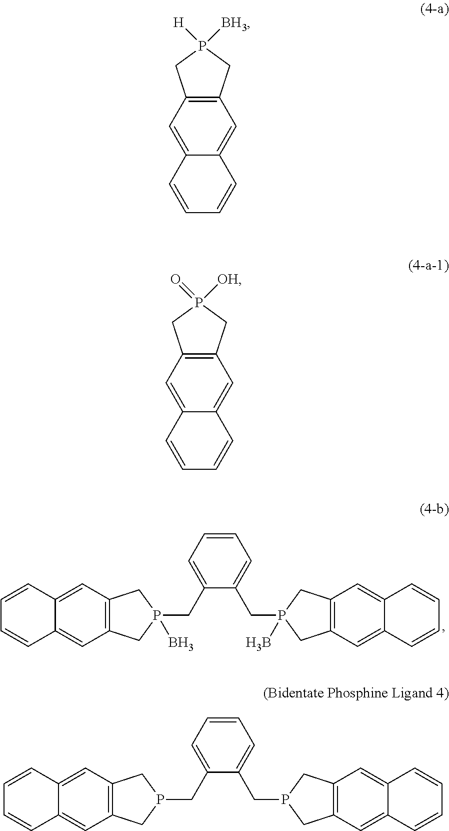

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (4-a-1), replacing 2,5-diphenylphosphane-borane complex (2-a) with an equimolar amount of a compound of formula (4-a), and replacing bis(2,5-diphenylphosphane-borane) (2-b) with an equimolar amount of a compound of formula (4-b), to finally afford bidentate phosphine ligand 4 as a colorless oily product (413 mg, 87%).

NMR data of bidentate phosphine ligand 4:

¹H NMR (400 MHz, CDCl₃): 7.72-7.64 (m, 2H), 7.57-7.48 (m, 3H), 7.18 (dd, J=5.9, 3.7 Hz, 1H), 7.03 (ddt, J=5.7, 3.6, 1.1 Hz, 1H), 3.41 (d, J=13.7 Hz, 3H), 3.04 (dd, J=13.7, 1.1 Hz, 2H).

¹³C NMR (100 MHz, CDCl₃): δ=137.81, 136.10, 132.45, 130.05, 127.32, 126.43, 126.38, 124.66, 40.86, 32.52.

³¹P NMR (162 MHz, CDCl₃): δ=42.53.

### Preparation Example 5

### Preparation of Bidentate Phosphine Ligand 5

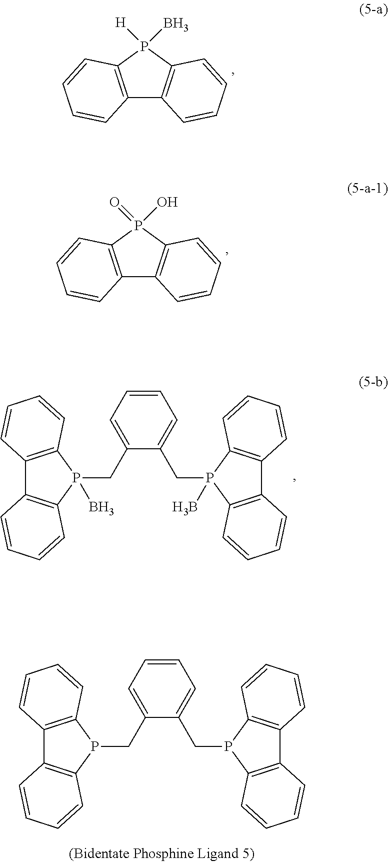

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (5-a-1), replacing 2,5-diphenylphosphane-borane complex (2-a) with an equimolar amount of a compound of formula (5-a), and replacing bis(2,5-diphenylphosphane-borane) (2-b) with an equimolar amount of a compound of formula (5-b), to finally afford bidentate phosphine ligand 5 as a colorless oily product (400 mg, 85%).

NMR data of bidentate phosphine ligand 5:

¹H NMR (400 MHz, CDCl₃): 7.83-7.75 (m, 2H), 7.65-7.57 (m, 2H), 7.48-7.38 (m, 4H), 7.19 (dd, J=5.9, 3.7 Hz, 1H), 7.04 (ddt, J=5.7, 3.7, 1.1 Hz, 1H), 3.73 (dd, J=13.6, 1.0 Hz, 2H).

¹³C NMR (100 MHz, CDCl₃): δ=144.25, 138.51, 138.29, 131.19, 130.03, 126.26, 126.15, 125.97, 125.76, 32.09.

³¹P NMR (162 MHz, CDCl₃): δ=41.62.

### Preparation Example 6

### Preparation of Bidentate Phosphine Ligand 6

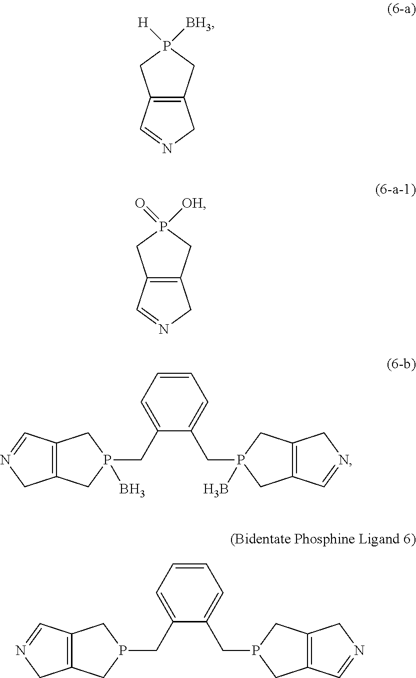

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (6-a-1), replacing 2,5-diphenylphosphane-borane complex (2-a) with an equimolar amount of a compound of formula (6-a), and replacing bis(2,5-diphenylphosphane-borane) (2-b) with an equimolar amount of a compound of formula (6-b), to finally afford bidentate phosphine ligand 6 as a colorless oily product (282 mg, 80%).

NMR data of bidentate phosphine ligand 6:

¹H NMR (400 MHz, CDCl₃): 7.43 (s, 1H), 7.15 (dd, J=5.9, 3.7 Hz, 1H), 7.10-7.03 (m, 1H), 4.26 (d, J=12.8 Hz, 1H), 4.19 (d, J=12.8 Hz, 1H), 3.21 (ddd, J=13.7, 12.8, 0.9 Hz, 1H), 3.11 (d, J=13.7 Hz, 2H), 2.96 (d, J=13.7 Hz, 2H), 2.86 (ddd, J=13.8, 12.9, 1.0 Hz, 1H).

¹³C NMR (100 MHz, CDCl₃): δ=151.13, 137.25, 130.92, 130.63, 129.98, 126.24, 58.79, 39.94, 39.89, 37.85, 31.53.

³¹P NMR (162 MHz, CDCl₃): δ=38.21.

### Preparation Example 7

### Preparation of Bidentate Phosphine Ligand 7

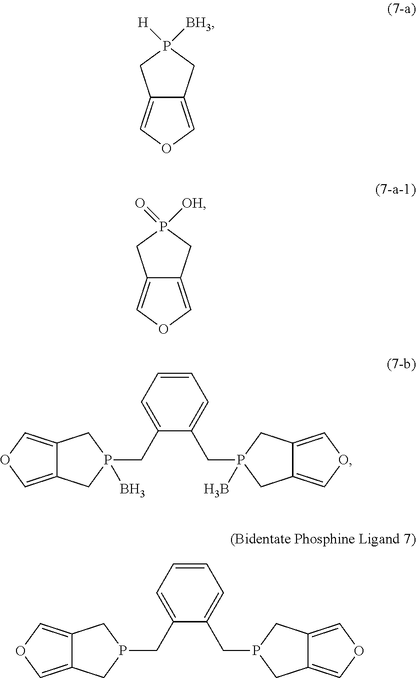

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (7-a-1), replacing 2,5-diphenylphosphane-borane complex (2-a) with an equimolar amount of a compound of formula (7-a), and replacing bis(2,5-diphenylphosphane-borane) (2-b) with an equimolar amount of a compound of formula (7-b), to finally afford bidentate phosphine ligand 7 as a colorless oily product (280 mg, 79%).

NMR data of bidentate phosphine ligand 7:

¹H NMR (400 MHz, CDCl₃): δ=7.17 (dd, J=5.9, 3.7 Hz, 1H), 7.09 (dddd, J=5.8, 3.6, 2.9, 1.0 Hz, 1H), 7.07 (s, 2H), 3.08 (dd, J=13.7, 4.6 Hz, 6H).

¹³C NMR (100 MHz, CDCl₃): δ=137.79, 137.55, 129.90, 129.44, 126.31, 36.37, 32.52.

³¹P NMR (162 MHz, CDCl₃): δ=39.77.

### Preparation Example 8

### Preparation of Bidentate Phosphine Ligand 8

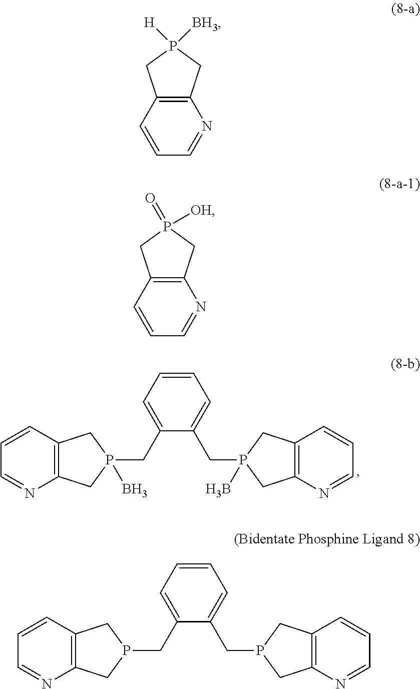

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (8-a-1), replacing 2,5-diphenylphosphane-borane complex (2-a) with an equimolar amount of a compound of formula (8-a), and replacing bis(2,5-diphenylphosphane-borane) (2-b) with an equimolar amount of a compound of formula (8-b), to finally afford bidentate phosphine ligand 8 as a colorless oily product (301 mg, 80%).

NMR data of bidentate phosphine ligand 8:

¹H NMR (400 MHz, CDCl₃): δ=8.49 (dd, J=3.5, 2.0 Hz, 1H), 7.44 (dd, J=7.8, 2.0 Hz, 1H), 7.34 (dd, J=7.7, 3.5 Hz, 1H), 7.16 (dd, J=5.8, 3.7 Hz, 1H), 7.02 (ddt, J=5.8, 3.7, 1.1 Hz, 1H), 3.64-3.55 (m, 1H), 3.37-3.17 (m, 3H), 3.10 (d, J=13.7 Hz, 2H).

¹³C NMR (100 MHz, CDCl₃): δ=159.46, 147.37, 137.76, 134.64, 132.23, 129.98, 126.37, 121.97, 42.05, 42.01, 40.09, 32.48.

³¹P NMR (162 MHz, CDCl₃): δ=40.93.

### Preparation Example 9

### Preparation of Bidentate Phosphine Ligand 9

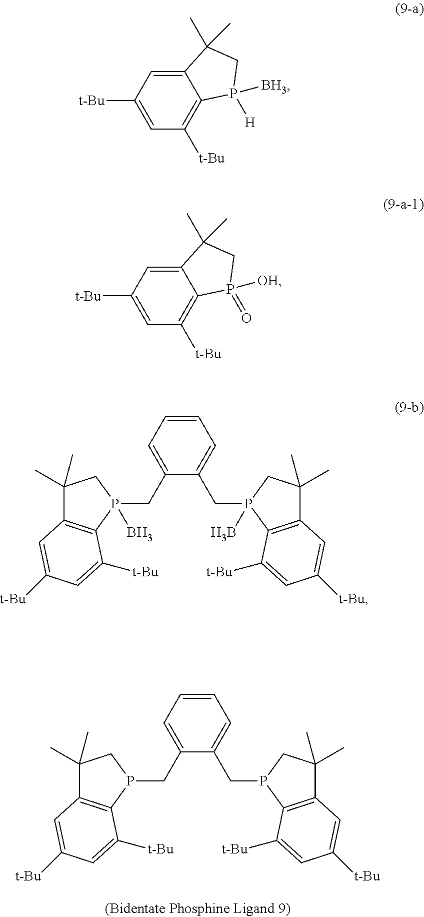

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (9-a-1), replacing 2,5-diphenylphosphane-borane complex (2-a) with an equimolar amount of a compound of formula (9-a), and replacing bis(2,5-diphenylphosphane-borane) (2-b) with an equimolar amount of a compound of formula (9-b), to finally afford bidentate phosphine ligand 9 as a colorless oily product (524 mg, 80%).

NMR data of bidentate phosphine ligand 9:

¹H NMR (400 MHz, CDCl₃): δ=7.12 (dd, J=6.1, 4.4 Hz, 1H), 7.00 (ddd, J=13.6, 4.1, 2.2 Hz, 2H), 6.91 (ddt, J=5.8, 4.4, 1.1 Hz, 1H), 3.52 (ddd, J=13.8, 12.9, 1.0 Hz, 1H), 3.19 (ddd, J=13.8, 12.8, 0.9 Hz, 1H), 2.53 (d, J=13.7 Hz, 1H), 1.36-1.28 (m, 21H).

¹³C NMR (100 MHz, CDCl₃): δ=153.29, 143.60, 137.52, 137.14, 135.65, 129.64, 126.35, 124.25, 121.73, 42.68, 36.14, 34.87, 34.48, 32.24, 31.44, 30.28, 29.59.

³¹P NMR (162 MHz, CDCl₃): δ=25.82.

### Preparation Example 10

### Preparation of Bidentate Phosphine Ligand 10

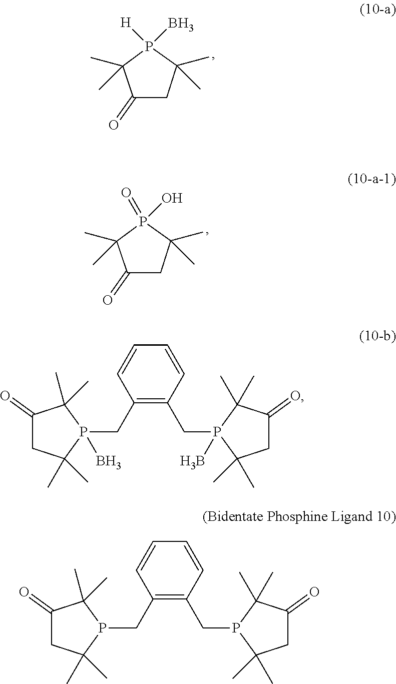

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (10-a-1), replacing 2,5-diphenylphosphane-borane complex (2-a) with an equimolar amount of a compound of formula (10-a), replacing bis(2,5-diphenylphosphane-borane) (2-b) with an equimolar amount of a compound of formula (10-b), to finally afford bidentate phosphine ligand 10 as a colorless oily product (356 mg, 85%).

NMR data of bidentate phosphine ligand 10:

¹H NMR (400 MHz, CDCl₃): δ=7.18 (dd, J=6.0, 4.4 Hz, 2H), 7.09-7.01 (m, 2H), 3.46 (td, J=13.6, 1.0 Hz, 2H), 3.01 (td, J=13.6, 1.0 Hz, 2H), 2.49 (s, 3H), 1.36 (s, 9H), 1.21 (s, 9H).

¹³C NMR (100 MHz, CDCl₃): δ=214.52, 135.46, 131.36, 126.31, 50.31, 40.00, 38.68, 27.59, 23.49, 23.47, 23.44, 23.42, 20.41.

³¹P NMR (162 MHz, CDCl₃): δ=15.02.

### Preparation Example 11

### Preparation of Bidentate Phosphine Ligand 11

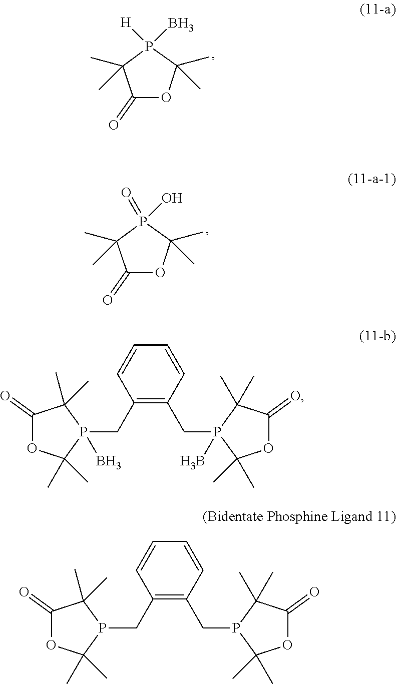

The ligand was prepared by the same method as described in Preparation Example 2, except for replacing 1-hydroxy-1-oxo-2,5-diphenylphosphine (2-a-1) in Preparation Example 2 with an amount of a compound of formula (11-a-1), replacing 2,5-diphenylphosphane-borane complex (2-a) with an equimolar amount of a compound of formula (11-a), and replacing bis(2,5-diphenylphosphane-borane) (2-b) with an equimolar amount of a compound of formula (11-b), to finally afford bidentate phosphine ligand 11 as a colorless oily product (355 mg, 84%).

NMR data of bidentate phosphine ligand 11:

¹H NMR (400 MHz, CDCl₃): δ=7.17 (dd, J=6.0, 4.4 Hz, 2H), 7.05 (ddt, J=6.6, 4.6, 1.0 Hz, 2H), 3.48 (td, J=13.6, 1.0 Hz, 2H), 3.03 (td, J=13.6, 1.0 Hz, 2H), 1.52 (s, 9H), 1.42 (s, 9H).

¹³C NMR (100 MHz, CDCl₃): δ=177.43, 135.37, 131.45, 126.38, 77.87, 49.53, 28.30, 22.60, 22.58, 22.55, 22.53, 20.85.

³¹P NMR (162 MHz, CDCl₃): δ=13.65.

### Preparation Example 12

### Preparation of Bidentate Phosphine Ligand 12

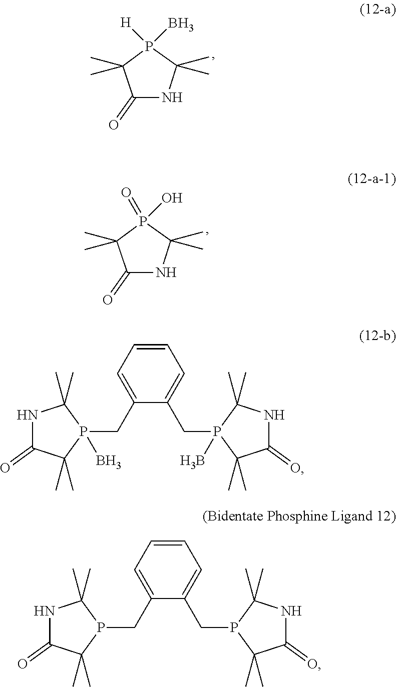

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (12-a-1), replacing 2,5-diphenylphosphane-borane complex (2-a) with an equimolar amount of a compound of formula (12-a), and replacing bis(2,5-diphenylphosphane-borane) (2-b) with an equimolar amount of a compound of formula (12-b), to finally afford bidentate phosphine ligand 12 as a colorless oily product (349 mg, 83%).

NMR data of bidentate phosphine ligand 12:

¹H NMR (400 MHz, CDCl₃): δ=7.17 (dd, J=5.9, 3.7 Hz, 2H), 7.04 (ddt, J=5.8, 3.7, 1.0 Hz, 2H), 6.19 (s, 1H), 3.33 (td, J=13.5, 1.0 Hz, 2H), 3.01 (td, J=13.5, 1.0 Hz, 2H), 1.45 (s, 9H), 1.40 (s, 9H).

¹³C NMR (100 MHz, CDCl₃): δ=177.81, 135.71, 131.44, 126.31, 68.67, 57.39, 28.58, 24.13, 21.56, 21.53, 21.51, 21.48.

³¹P NMR (162 MHz, CDCl₃): δ=11.77.

### Preparation Example 13

### Preparation of Bidentate Phosphine Ligand 13

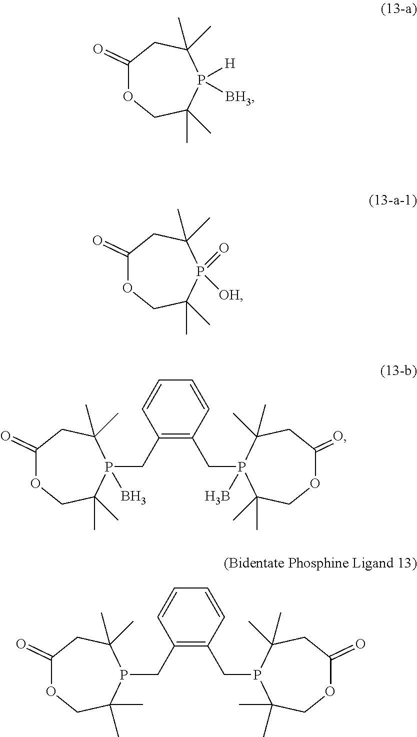

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (13-a-1), replacing 2,5-diphenylphosphane-borane complex (2-a) with an equimolar amount of a compound of formula (13-a), and replacing bis(2,5-diphenylphosphane-borane) (2-b) with an equimolar amount of a compound of formula (13-b), to finally afford bidentate phosphine ligand 13 as a colorless oily product (383 mg, 80%).

NMR data of bidentate phosphine ligand 13:

¹H NMR (400 MHz, CDCl₃): δ=7.16 (dd, J=6.0, 4.4 Hz, 1H), 7.07-6.99 (m, 1H), 4.04 (s, 2H), 3.39 (td, J=13.5, 1.0 Hz, 1H), 2.92 (td, J=13.5, 1.0 Hz, 1H), 2.35 (s, 2H), 1.18 (d, J=8.0 Hz, 14H).

¹³C NMR (100 MHz, CDCl₃): δ=173.06, 136.83, 131.47, 126.34, 68.72, 46.76, 41.62, 41.13, 27.58, 23.72, 23.69, 23.66, 23.63, 21.76.

³¹P NMR (162 MHz, CDCl₃): δ=43.13.

### Preparation Example 14

### Preparation of Bidentate Phosphine Ligand 14

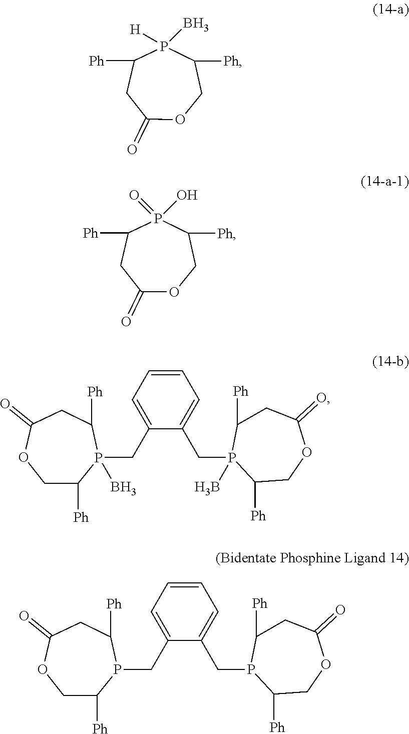

The ligand was prepared by the same method as described in Preparation Example 2, except for 1-hydroxy-1-oxo-2,5-diphenylphosphine (2-a-1) in Preparation Example 2 with an equimolar amount of a compound of formula (14-a-1), replacing 2,5-diphenylphosphane-borane complex (2-a) with an equimolar amount of a compound of formula (14-a), and replacing bis(2,5-diphenylphosphane-borane) (2-b) with an equimolar amount of a compound of formula (14-b), to finally afford bidentate phosphine ligand 14 as a colorless oily product (530 mg, 79%).

NMR data of bidentate phosphine ligand 14:

¹H NMR (400 MHz, CDCl₃): δ=7.40-7.32 (m, 2H), 7.31-7.17 (m, 6H), 7.16-7.09 (m, 2H), 7.07 (dd, J=5.9, 3.7 Hz, 1H), 6.94 (ddt, J=5.7, 3.6, 1.0 Hz, 1H), 4.59 (dd, J=9.4, 5.0 Hz, 1H), 4.30 (dd, J=9.4, 5.0 Hz, 1H), 3.76 (tt, J=6.4, 1.0 Hz, 1H), 3.35-3.20 (m, 3H), 3.17 (dd, J=14.8, 6.4 Hz, 1H), 2.92 (dd, J=14.8, 6.4 Hz, 1H).

¹³C NMR (100 MHz, CDCl₃): δ=172.74, 139.33, 139.29, 139.20, 139.14, 130.17, 130.16, 130.15, 130.15, 130.11, 129.54, 128.19, 128.11, 128.09, 126.46, 126.40, 126.39, 126.37, 126.36, 65.94, 47.68, 47.44, 39.02, 32.23.

³¹P NMR (162 MHz, CDCl₃): δ=46.50.

### Preparation Example 15

### Preparation of Bidentate Phosphine Ligand 15

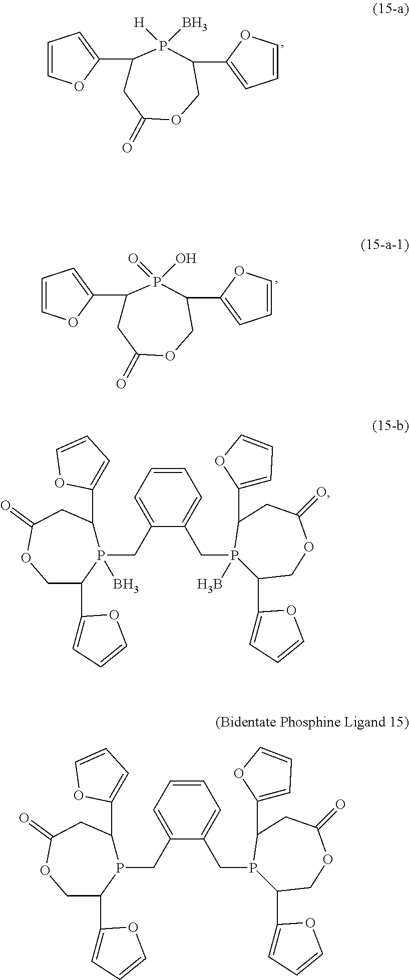

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (15-a-1), replacing 2,5-diphenylphosphane-borane complex (2-a) with an equimolar amount of a compound of formula (15-a), and replacing bis(2,5-diphenylphosphane-borane) (2-b) with an equimolar amount of a compound of formula (15-b), to finally afford bidentate phosphine ligand 15 as a colorless oily product (492 mg, 78%).

NMR data of bidentate phosphine ligand 15:

¹H NMR (400 MHz, CDCl₃): δ=7.38 (dt, J=18.3, 1.5 Hz, 2H), 7.21 (dd, J=5.9, 3.8 Hz, 1H), 7.14 (ddt, J=6.5, 3.8, 1.0 Hz, 1H), 6.37 (ddd, J=17.5, 7.8, 1.5 Hz, 2H), 6.20 (ddd, J=7.9, 4.0, 1.5 Hz, 2H), 4.87 (dd, J=9.4, 5.0 Hz, 1H), 4.72 (t, J=6.4 Hz, 1H), 4.54 (dd, J=9.5, 5.1 Hz, 1H), 3.99 (t, J=5.0 Hz, 1H), 3.75 (ddd, J=13.9, 13.1, 1.0 Hz, 1H), 3.26 (dd, J=14.6, 6.4 Hz, 1H), 3.15 (ddd, J=13.8, 13.0, 0.9 Hz, 1H), 3.01 (dd, J=14.8, 6.4 Hz, 1H).

¹³C NMR (100 MHz, CDCl₃): δ=172.72, 157.14, 157.05, 157.03, 143.77, 139.10, 129.50, 126.41, 111.55, 111.52, 108.50, 107.82, 107.80, 64.62, 44.13, 38.83, 37.83, 31.98.

³¹P NMR (162 MHz, CDCl₃): δ=44.25.

### Preparation Example 16

### Preparation of Bidentate Phosphine Ligand 16

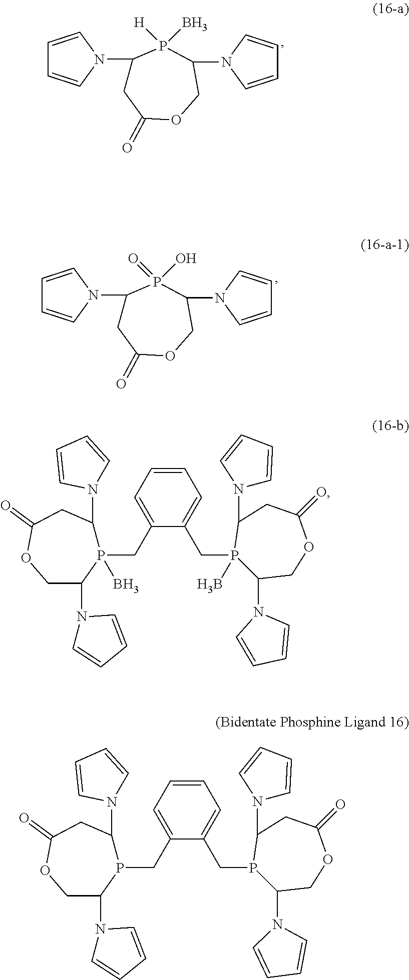

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (16-a-1), replacing 2,5-diphenylphosphane-borane complex (2-a) with an equimolar amount of a compound of formula (16-a), and replacing bis(2,5-diphenylphosphane-borane) (2-b) with an equimolar amount of a compound of formula (16-b), to finally afford bidentate phosphine ligand 16 as a colorless oily product (489 mg, 78%).

NMR data of bidentate phosphine ligand 16:

¹H NMR (400 MHz, CDCl₃): δ=7.20-7.12 (m, 5H), 7.06 (ddt, J=5.8, 3.8, 1.1 Hz, 1H), 6.03 (td, J=4.2, 1.3 Hz, 4H), 4.84 (t, J=4.9 Hz, 1H), 4.73 (t, J=6.3 Hz, 1H), 4.62 (dd, J=8.2, 4.9 Hz, 1H), 4.31 (dd, J=8.2, 4.9 Hz, 1H), 3.45 (ddd, J=13.8, 13.0, 0.9 Hz, 1H), 3.05-2.95 (m, 2H), 2.92 (dd, J=13.7, 6.2 Hz, 1H).

¹³C NMR (100 MHz, CDCl₃): δ=172.76, 139.26, 129.42, 126.42, 122.78, 122.72, 122.67, 122.61, 122.44, 110.05, 110.04, 110.02, 110.01, 109.94, 66.44, 63.51, 58.28, 37.95, 29.96.

³¹P NMR (162 MHz, CDCl₃): δ=45.17.

### Preparation Example 17

### Preparation of Bidentate Phosphine Ligand 17

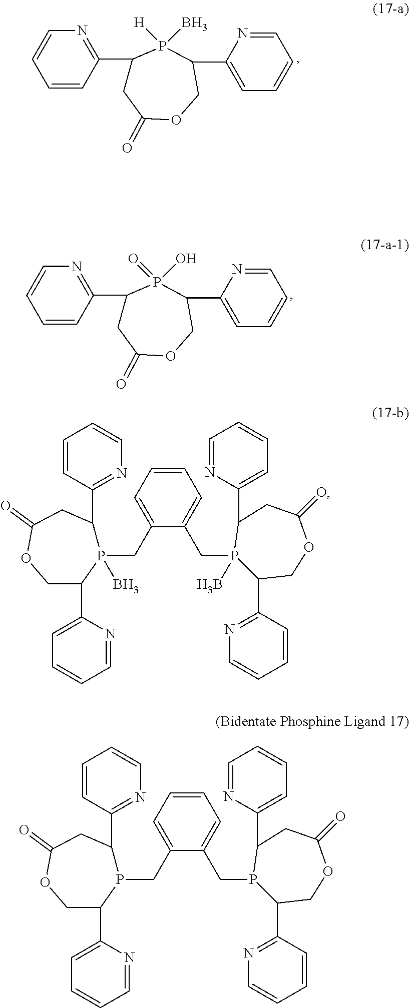

The ligand was prepared by the same method as described in Preparation Example 2, except for replacing 1-hydroxy-1-oxo-2,5-diphenylphosphine (2-a-1) in Preparation Example 2 with an equimolar of a compound of formula (17-a-1), replacing 2,5-diphenylphosphane-borane complex (2-a) with an equimolar amount of a compound of formula (17-a), and replacing bis(2,5-diphenylphosphane-borane) (2-b) with an equimolar amount of a compound of formula (17-b), to finally afford bidentate phosphine ligand 17 as a colorless oily product (533 mg, 79%).

NMR data of bidentate phosphine ligand 17:

¹H NMR (400 MHz, CDCl₃): δ=8.66 (ddd, J=4.8, 3.5, 1.5 Hz, 2H), 7.64 (tdd, J=7.7, 2.7, 1.6 Hz, 2H), 7.25 (ddd, J=12.9, 7.8, 1.4 Hz, 2H), 7.24-7.16 (m, 3H), 7.06 (ddt, J=5.7, 3.6, 1.0 Hz, 1H), 5.01 (dd, J=9.4, 5.0 Hz, 1H), 4.49 (dd, J=9.4, 5.0 Hz, 1H), 4.00 (t, J=6.4 Hz, 1H), 3.52 (dqd, J=14.1, 13.0, 1.0 Hz, 2H), 3.34 (dd, J=14.8, 6.4 Hz, 1H), 3.26 (t, J=5.0 Hz, 1H), 3.01 (dd, J=14.8, 6.4 Hz, 1H).

¹³C NMR (100 MHz, CDCl₃): δ=172.27, 160.28, 160.25, 160.04, 150.38, 139.18, 137.95, 137.93, 137.91, 129.57, 126.37, 122.23, 122.19, 122.17, 121.17, 121.17, 121.01, 64.86, 56.28, 49.35, 38.28, 32.21.

³¹P NMR (162 MHz, CDCl₃): δ=47.98.

### Preparation Example 18

### Preparation of Bidentate Phosphine Ligand 18

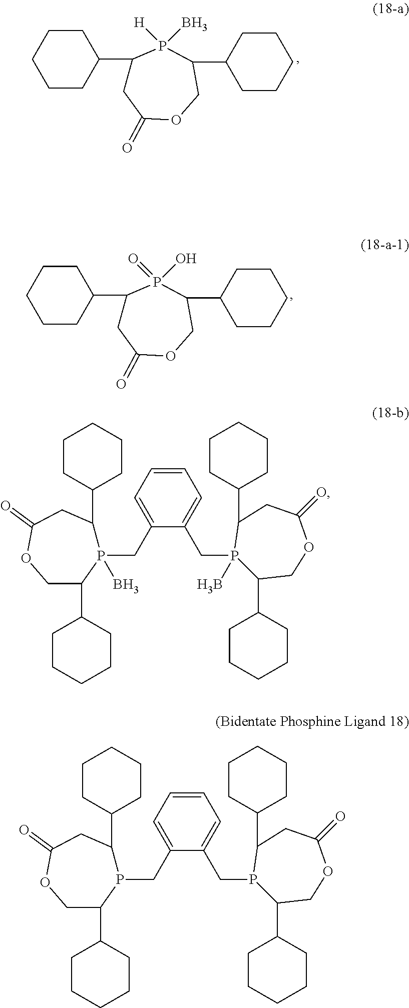

The ligand was prepared by the same method as described in Preparation Example 2, except for replacing 1-hydroxy-1-oxo-2,5-diphenylphosphine (2-a-1) in Preparation Example 2 equimolar amount of a compound of formula (18-a-1), replacing 2,5-diphenylphosphane-borane complex (2-a) with an equimolar amount of a compound of formula (18-a), and replacing bis(2,5-diphenylphosphane-borane) (2-b) with an equimolar amount of a compound of formula (18-b), to finally afford bidentate phosphine ligand 18 as a colorless oily product (521 mg, 75%).

NMR data of bidentate phosphine ligand 18:

¹H NMR (400 MHz, CDCl₃): δ=7.14 (t, J=4.5 Hz, 1H), 7.09-7.03 (m, 1H), 4.35-4.27 (m, 1H), 4.03-3.95 (m, 1H), 3.35 (ddd, J=13.7, 12.4, 1.0 Hz, 1H), 2.94 (ddd, J=12.3, 7.6, 1.4 Hz, 1H), 2.80 (ddd, J=13.5, 12.4, 1.0 Hz, 1H), 2.67 (ddd, J=12.3, 7.6, 1.4 Hz, 1H), 2.27-2.17 (m, 1H), 1.74-1.42 (m, 16H), 1.34-1.11 (m, 11H).

¹³C NMR (100 MHz, CDCl₃): δ=173.37, 139.17, 129.54, 126.45, 63.85, 43.33, 41.78, 41.76, 41.71, 37.23, 31.51, 29.94, 27.74, 27.68, 27.63, 27.58, 27.16, 26.29, 26.28, 26.26, 26.25, 26.00, 25.96, 25.93, 25.89, 25.78.

³¹P NMR (162 MHz, CDCl₃): δ=36.33.

### Preparation Example 19

### Preparation of Bidentate Phosphine Ligand 19

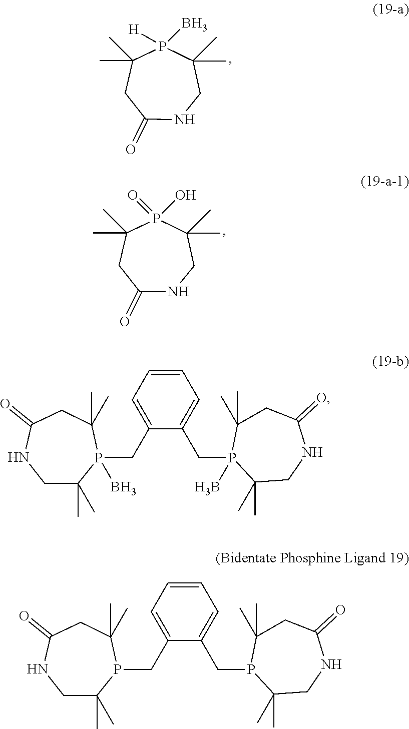

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (19-a-1), replacing 2,5-diphenylphosphane-borane complex (2-a) with an equimolar amount of a compound of formula (19-a), and replacing bis(2,5-diphenylphosphane-borane) (2-b) with an equimolar amount of a compound of formula (19-b), to finally afford bidentate phosphine ligand 19 as a colorless oily product (381 mg, 80%).

NMR data of bidentate phosphine ligand 19:

¹H NMR (400 MHz, CDCl₃): δ=7.17 (dd, J=5.9, 3.7 Hz, 1H), 7.03 (ddt, J=5.8, 3.7, 1.0 Hz, 1H), 6.73 (t, J=5.3 Hz, 1H), 3.35-3.25 (m, 3H), 2.93 (td, J=13.6, 1.0 Hz, 1H), 2.25 (s, 1H), 1.18 (d, J=6.2 Hz, 12H).

¹³C NMR (100 MHz, CDCl₃): δ=172.94, 136.70, 131.45, 126.34, 47.30, 41.66, 41.53, 38.89, 27.50, 23.72, 22.61, 22.57, 22.54, 22.51.

³¹P NMR (162 MHz, CDCl₃): δ=40.87.

### Preparation Example 20

### Preparation of Bidentate Phosphine Ligand 20

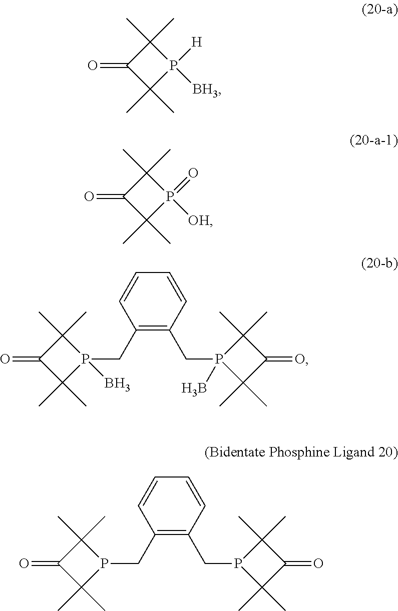

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (20-a-1), replacing 2,5-diphenylphosphane-borane complex (2-a) with an equimolar amount of a compound of formula (20-a), and replacing bis(2,5-diphenylphosphane-borane) (2-b) with an equimolar amount of a compound of formula (20-b), to finally afford bidentate phosphine ligand 20 as a colorless oily product (324 mg, 83%).

NMR data of bidentate phosphine ligand 20:

¹H NMR (400 MHz, CDCl₃): δ=7.17 (dd, J=6.0, 4.4 Hz, 1H), 7.06 (ddt, J=6.6, 4.4, 1.0 Hz, 1H), 3.23 (dd, J=13.7, 1.0 Hz, 2H), 1.40 (s, 9H).

¹³C NMR (100 MHz, CDCl₃): δ=207.63, 131.24, 130.75, 126.30, 50.77, 26.94, 22.17.

³¹P NMR (162 MHz, CDCl₃): δ=58.56.

### Preparation Example 21

### Preparation of Bidentate Phosphine Ligand 21

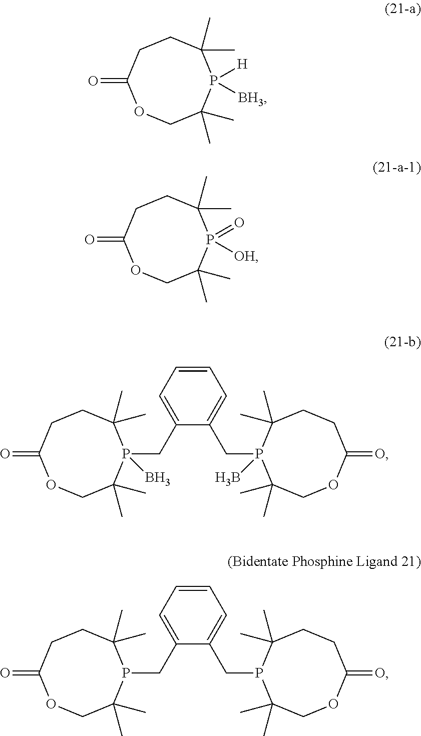

The ligand was prepared by the same method as described in Preparation Example 2, except for 1-hydroxy-1-oxo-2,5-diphenylphosphine (2-a-1) in Preparation Example 2 with an equimolar amount of a compound of formula (21-a-1), replacing 2,5-diphenylphosphane-borane complex (2-a) with an equimolar amount of a compound of formula (21-a), and replacing bis(2,5-diphenylphosphane-borane) (2-b) with an equimolar amount of a compound of formula (21-b), to finally afford bidentate phosphine ligand 21 as a colorless oily product (405 mg, 80%).

NMR data of bidentate phosphine ligand 21:

¹H NMR (400 MHz, CDCl₃): δ=7.17 (dd, J=5.8, 3.7 Hz, 2H), 7.04 (ddt, J=5.7, 3.6, 1.0 Hz, 2H), 4.04 (s, 3H), 3.35 (td, J=13.4, 1.0 Hz, 2H), 2.89 (td, J=13.5, 1.0 Hz, 2H), 2.33 (t, J=8.0 Hz, 4H), 1.69 (t, J=8.1 Hz, 4H), 1.18 (s, 9H), 1.12 (s, 9H).

¹³C NMR (100 MHz, CDCl₃): δ=172.37, 136.83, 131.21, 126.44, 69.41, 45.77, 38.67, 33.36, 31.69, 27.91, 23.60, 21.84, 21.81, 21.77, 21.74.

³¹P NMR (162 MHz, CDCl₃): δ=36.39.

### Preparation Example 22

### Preparation of Bidentate Phosphine Ligand 22

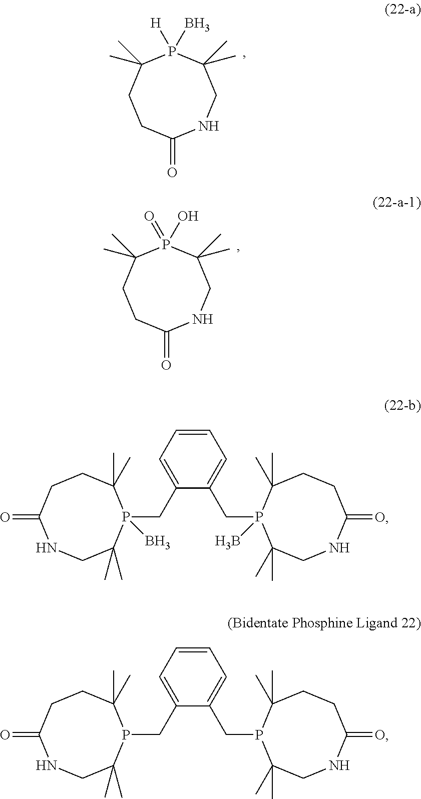

The ligand was prepared by the same method as described in Preparation Example 2, except for replacing 1-hydroxy-1-oxo-2,5-diphenylphosphine (2-a-1) in Preparation Example 2 with an equimolar of a compound of formula (22-a-1), replacing 2,5-diphenylphosphane-borane complex (2-a) with an equimolar amount of a compound of formula (22-a), and replacing bis(2,5-diphenylphosphane-borane) (2-b) with an equimolar amount of a compound of formula (22-b), to finally afford bidentate phosphine ligand 22 as a colorless oily product (394 mg, 78%).

NMR data of bidentate phosphine ligand 22:

¹H NMR (400 MHz, CDCl₃): δ=7.17 (dd, J=5.9, 3.7 Hz, 1H), 7.04 (ddt, J=5.7, 3.6, 1.0 Hz, 1H), 6.73 (t, J=5.3 Hz, 1H), 3.37 (dd, J=12.7, 5.4 Hz, 1H), 3.35-3.26 (m, 2H), 2.96 (td, J=13.4, 0.9 Hz, 1H), 2.28 (t, J=8.0 Hz, 2H), 1.61 (t, J=8.1 Hz, 2H), 1.18 (s, 4H), 1.10 (s, 4H).

¹³C NMR (100 MHz, CDCl₃): δ=173.56, 136.80, 131.21, 126.44, 51.18, 39.01, 38.60, 33.87, 33.02, 27.83, 23.73, 23.70, 23.67, 23.64, 22.50.

³¹P NMR (162 MHz, CDCl₃): δ=34.25.

### Preparation Example 23

### Preparation of Bidentate Phosphine Ligand 23

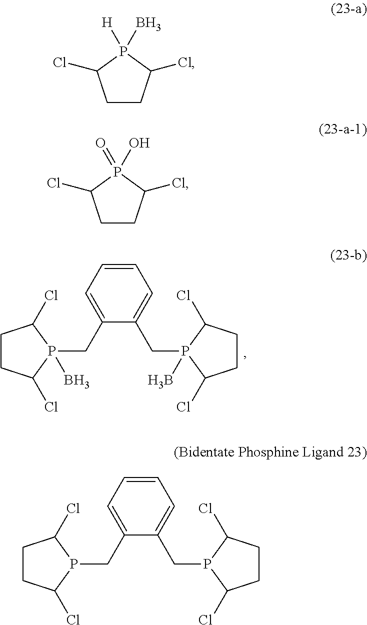

The ligand was prepared by the same method as described in Preparation Example 2, except for replacing 1-hydroxy-1-oxo-2,5-diphenylphosphine (2-a-1) in Preparation Example 2 with an equimolar amount f a compound of formula (23-a-1), replacing 2,5-diphenylphosphane-borane complex (2-a) with an equimolar amount of a compound of formula (23-a), and replacing bis(2,5-diphenylphosphane-borane) (2-b) with an equimolar amount of a compound of formula (23-b), to finally afford bidentate phosphine ligand 23 as a colorless oily product (312 mg, 75%).

NMR data of bidentate phosphine ligand 23:

¹H NMR (400 MHz, CDCl₃): δ=7.17 (dd, J=5.8, 3.6 Hz, 1H), 7.04 (ddt, J=5.7, 3.6, 1.0 Hz, 1H), 4.09-4.00 (m, 2H), 3.45-3.35 (m, 1H), 2.98 (td, J=13.4, 13.0, 1.1 Hz, 1H), 2.21-2.08 (m, 4H).

¹³C NMR (100 MHz, CDCl₃): δ=138.21, 138.16, 138.13, 138.08, 129.45, 129.42, 129.41, 129.37, 126.23, 126.20, 66.26, 66.07, 31.05, 30.95, 30.34, 30.30, 30.15, 30.12.

³¹P NMR (162 MHz, CDCl₃): δ=40.25.

### Preparation Example 24

### Preparation of Bidentate Phosphine Ligand 24

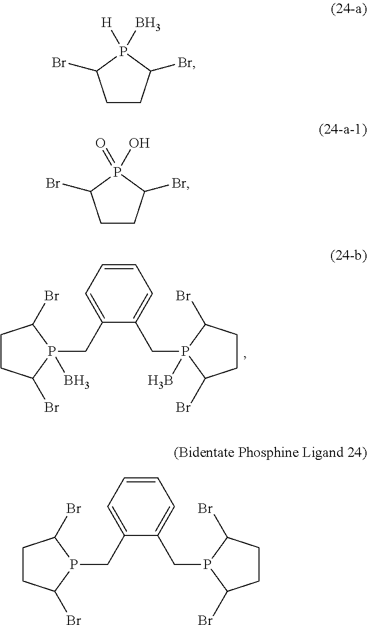

The ligand was prepared by the same method as described in Preparation Example 2, except for replacing 1-hydroxy-1-oxo-2,5-diphenylphosphine (2-a-1) in Preparation Example 2 of a compound of formula (24-a-1), replacing with an equimolar amount 2,5-diphenylphosphane-borane complex (2-a) with an equimolar amount of a compound of formula (24-a), and replacing bis(2,5-diphenylphosphane-borane) (2-b) with an equimolar amount of a compound of formula (24-b), to finally afford bidentate phosphine ligand 24 as a colorless oily product (451 mg, 76%).

NMR data of bidentate phosphine ligand 24:

¹H NMR (400 MHz, CDCl₃): δ=7.16 (dd, J=5.9, 3.8 Hz, 1H), 7.10 (dddd, J=5.8, 3.7, 2.9, 1.0 Hz, 1H), 3.89 (dddd, J=5.7, 3.7, 2.4, 1.3 Hz, 2H), 3.46-3.37 (m, 1H), 3.18 (ddd, J=13.8, 12.9, 0.9 Hz, 1H), 2.34-2.20 (m, 4H).

¹³C NMR (100 MHz, CDCl₃): δ=137.92, 137.88, 137.84, 137.79, 129.37, 129.33, 129.32, 129.29, 126.25, 126.22, 51.82, 51.64, 31.21, 31.18, 31.12, 31.02, 31.00.

³¹P NMR (162 MHz, CDCl₃): δ=43.78.

### Preparation Example 25

### Preparation of Bidentate Phosphine Ligand 25

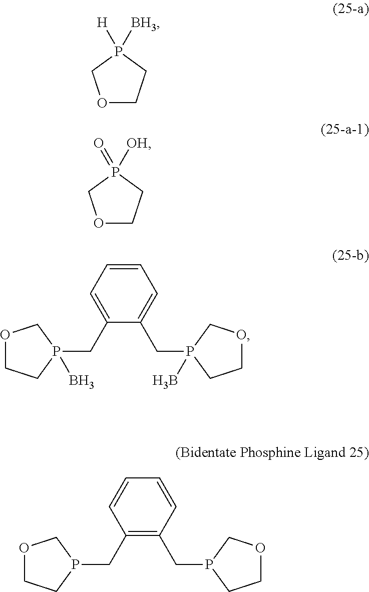

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (25-a-1), replacing 2,5-diphenylphosphane-borane complex (2-a) with an equimolar amount of a compound of formula (25-a), and replacing bis(2,5-diphenylphosphane-borane) (2-b) with an equimolar amount of a compound of formula (25-b), to finally afford bidentate phosphine ligand 25 as a colorless oily product (234 mg, 83%).

NMR data of bidentate phosphine ligand 25:

¹H NMR (400 MHz, CDCl₃): δ=7.16 (dd, J=5.8, 3.7 Hz, 1H), 7.04 (ddt, J=5.8, 3.7, 1.0 Hz, 1H), 3.80-3.68 (m, 2H), 3.46 (d, J=13.7 Hz, 1H), 3.13 (ddd, J=13.7, 12.8, 0.9 Hz, 1H), 2.83 (ddd, J=13.8, 12.8, 0.9 Hz, 1H), 2.38 (dt, J=13.7, 4.6 Hz, 2H).

¹³C NMR (100 MHz, CDCl₃): δ=138.00, 137.96, 137.92, 137.88, 129.95, 129.92, 129.90, 129.87, 126.26, 126.23, 69.41, 69.22, 65.42, 65.33, 30.47, 30.44, 30.29, 30.25, 26.79, 26.60.

³¹P NMR (162 MHz, CDCl₃): δ=32.11.

### Preparation Example 26

### Preparation of Bidentate Phosphine Ligand 26

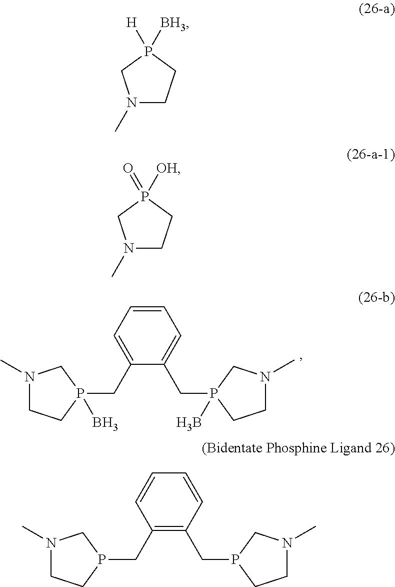

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (26-a-1), replacing 2,5-diphenylphosphane-borane complex (2-a) with an equimolar amount of a compound of formula (26-a), and replacing bis(2,5-diphenylphosphane-borane) (2-b) with an equimolar amount of a compound of formula (26-b), to finally afford bidentate phosphine ligand 26 as a colorless oily product (250 mg, 81%).

NMR data of bidentate phosphine ligand 26:

¹H NMR (400 MHz, CDCl₃): δ=7.15 (dd, J=5.9, 3.7 Hz, 1H), 7.06 (ddt, J=5.8, 3.8, 1.1 Hz, 1H), 3.12 (ddd, J=13.8, 12.9, 1.1 Hz, 1H), 3.01 (ddd, J=11.0, 4.4, 3.4 Hz, 1H), 2.92-2.79 (m, 2H), 2.54 (d, J=13.5 Hz, 1H), 2.32 (s, 2H), 2.25 (tdd, J=13.4, 4.3, 3.4 Hz, 1H), 1.80 (tdd, J=13.4, 4.4, 3.3 Hz, 1H).

¹³C NMR (100 MHz, CDCl₃): δ=137.45, 129.78, 126.27, 58.14, 50.05, 40.55, 30.64, 27.66.

³¹P NMR (162 MHz, CDCl₃): δ=33.26.

### Preparation Example 27

### Preparation of Bidentate Phosphine Ligand 27

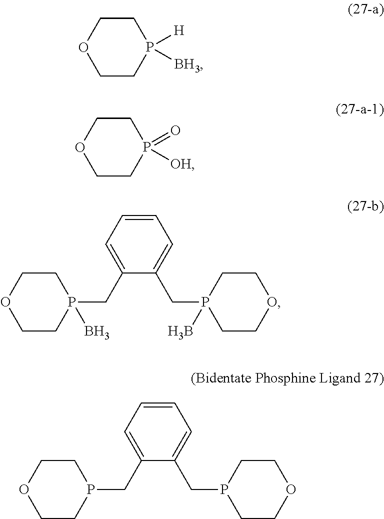

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (27-a-1), replacing 2,5-diphenylphosphane-borane complex (2-a) with an equimolar amount of a compound of formula (27-a), and replacing bis(2,5-diphenylphosphane-borane) (2-b) with an equimolar amount of a compound of formula (27-b), to finally afford bidentate phosphine ligand 27 as a colorless oily product (245 mg, 79%).

NMR data of bidentate phosphine ligand 27:

¹H NMR (400 MHz, CDCl₃): δ=7.15 (dd, J=5.9, 3.7 Hz, 1H), 7.04 (ddt, J=5.8, 3.7, 1.0 Hz, 1H), 3.54-3.43 (m, 4H), 2.93 (dd, J=13.7, 1.0 Hz, 2H), 1.78 (dt, J=13.7, 5.6 Hz, 4H).

¹³C NMR (100 MHz, CDCl₃): δ=137.67, 137.63, 137.59, 137.55, 130.06, 130.03, 130.01, 129.98, 126.22, 126.19, 66.01, 65.92, 31.65, 31.61, 31.46, 31.43, 27.05, 26.87.

³¹P NMR (162 MHz, CDCl₃): δ=46.74.

### Preparation Example 28

### Preparation of Bidentate Phosphine Ligand 28

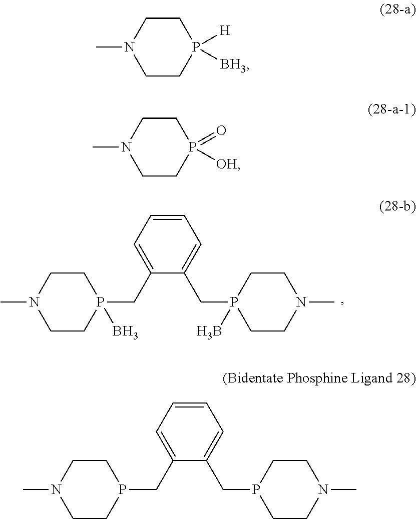

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (28-a-1), replacing 2,5-diphenylphosphane-borane complex (2-a) with an equimolar amount of a compound of formula (28-a), and replacing bis(2,5-diphenylphosphane-borane) (2-b) with an equimolar amount of a compound of formula (28-b), to finally afford bidentate phosphine ligand 28 as a colorless oily product (269 mg, 80%).

NMR data of bidentate phosphine ligand 28:

¹H NMR (400 MHz, CDCl₃): δ=7.15 (dd, J=5.9, 3.7 Hz, 1H), 7.03 (ddt, J=5.7, 3.6, 0.9 Hz, 1H), 2.91 (dd, J=13.7, 1.1 Hz, 2H), 2.50 (dd, J=5.3, 4.6 Hz, 4H), 2.27 (s, 2H), 1.71-1.62 (m, 4H).

¹³C NMR (100 MHz, CDCl₃): δ=137.46, 129.74, 126.23, 50.48, 45.48, 31.46, 26.82.

³¹P NMR (162 MHz, CDCl₃): δ=51.68.

### Preparation Example 29

### Preparation of Bidentate Phosphine Ligand 29

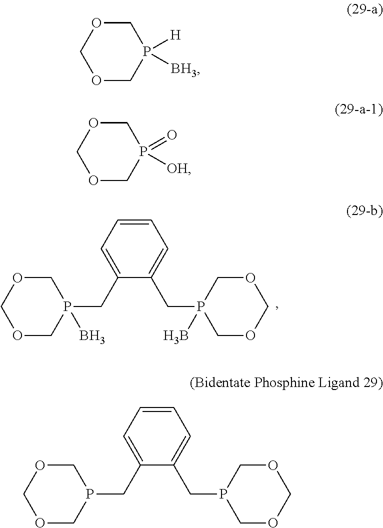

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (29-a-1), replacing 2,5-diphenylphosphane-borane complex (2-a) with an equimolar amount of a compound of formula (29-a), and replacing bis(2,5-diphenylphosphane-borane) (2-b) with an equimolar amount of a compound of formula (29-b), to finally afford bidentate phosphine ligand 29 as a colorless oily product (245 mg, 78%).

NMR data of bidentate phosphine ligand 29:

¹H NMR (400 MHz, CDCl₃): δ=7.17 (dd, J=5.9, 3.7 Hz, 1H), 6.97 (ddt, J=5.7, 3.7, 1.0 Hz, 1H), 4.68 (d, J=2.9 Hz, 1H), 4.42 (d, J=2.7 Hz, 1H), 3.49 (d, J=13.7 Hz, 3H), 3.00 (dd, J=13.7, 0.9 Hz, 2H).

¹³C NMR (100 MHz, CDCl₃): δ=137.94, 137.89, 137.86, 137.81, 129.97, 129.94, 129.92, 129.89, 126.20, 126.16, 96.84, 96.77, 65.96, 65.77, 30.29, 30.25, 30.10, 30.07.

³¹P NMR (162 MHz, CDCl₃): δ=48.63.

### Preparation Example 30

### Preparation of Bidentate Phosphine Ligand 30

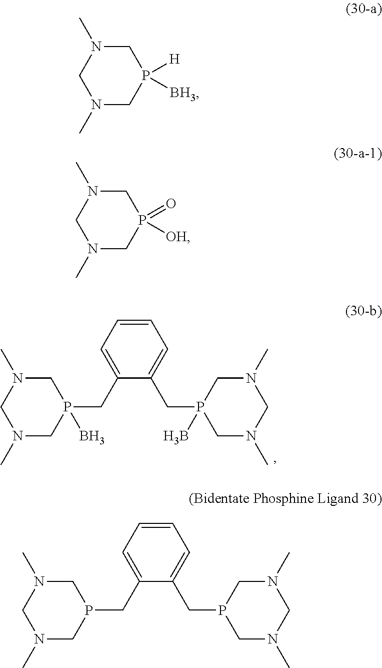

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (30-a-1), replacing 2,5-diphenylphosphane-borane complex (2-a) with an equimolar amount of a compound of formula (30-a), and replacing bis(2,5-diphenylphosphane-borane) (2-b) with an equimolar amount of a compound of formula (30-b), to finally afford bidentate phosphine ligand 30 as a colorless oily product (286 mg, 78%).

NMR data of bidentate phosphine ligand 30:

¹H NMR (400 MHz, CDCl₃): δ=7.16 (dd, J=5.9, 3.7 Hz, 2H), 7.03 (ddt, J=5.8, 3.8, 1.0 Hz, 2H), 3.18 (s, 3H), 2.97 (dd, J=13.7, 1.0 Hz, 4H), 2.62 (d, J=13.5 Hz, 6H), 2.30 (s, 9H).

¹³C NMR (100 MHz, CDCl₃): δ=136.99, 129.87, 126.28, 74.81, 54.82, 43.68, 32.24.

³¹P NMR (162 MHz, CDCl₃): δ=53.95.

### Preparation Example 31

### Preparation of Bidentate Phosphine Ligand 31

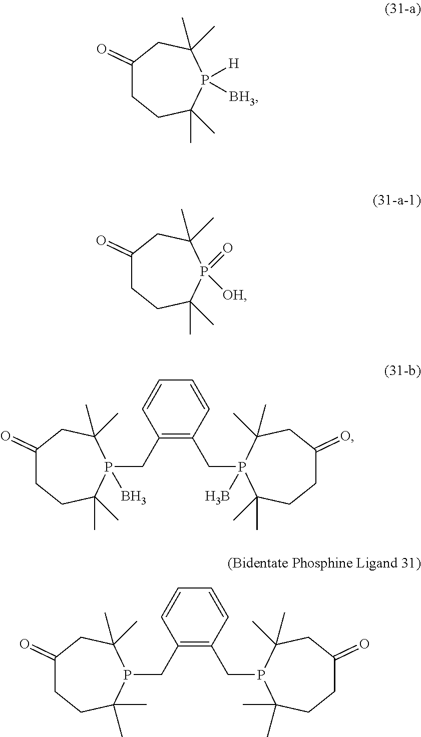

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (31-a-1), replacing 2,5-diphenylphosphane-borane complex (2-a) with an equimolar amount of a compound of formula (31-a), and replacing bis(2,5-diphenylphosphane-borane) (2-b) with an equimolar amount of a compound of formula (31-b), to finally afford bidentate phosphine ligand 31 as a colorless oily product (389 mg, 82%).

NMR data of bidentate phosphine ligand 31:

¹H NMR (400 MHz, CDCl₃): δ=7.18 (dd, J=6.1, 4.4 Hz, 1H), 7.08-7.00 (m, 1H), 3.36 (td, J=13.5, 1.0 Hz, 1H), 2.93 (td, J=13.4, 0.9 Hz, 1H), 2.49-2.37 (m, 3H), 1.65-1.50 (m, 2H), 1.18 (s, 4H), 1.12 (s, 4H).

¹³C NMR (100 MHz, CDCl₃): δ=213.65, 136.74, 131.45, 126.37, 43.79, 43.70, 42.33, 40.29, 38.78, 33.04, 27.66, 23.74, 23.71, 23.68, 23.64, 23.53.

³¹P NMR (162 MHz, CDCl₃): δ=48.98.

### Preparation Example 32

### Preparation of Bidentate Phosphine Ligand 32

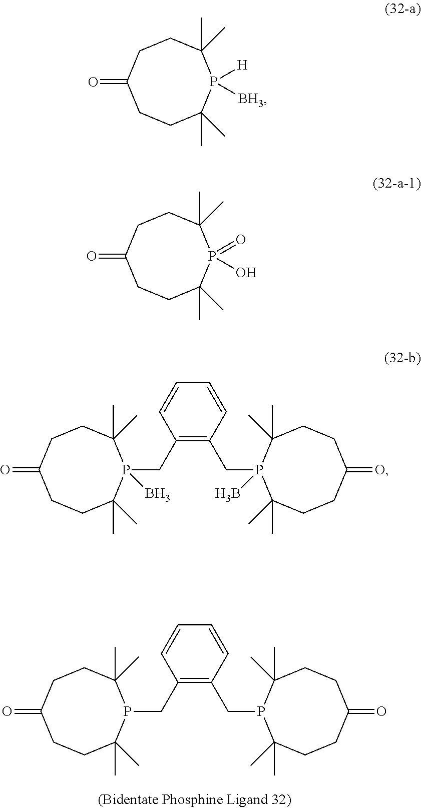

The ligand was prepared by the same method as described in Preparation Example 2, except for replacing 1-hydroxy-1-oxo-2,5-diphenylphosphine (2-a-1) in Preparation Example 2 with an equimolar amount of a compound of formula (32-a-1), replacing 2,5-diphenylphosphane-borane complex (2-a) with an equimolar amount of a compound of formula (32-a), and replacing bis(2,5-diphenylphosphane-borane) (2-b) with an equimolar amount of a compound of formula (32-b), to finally afford bidentate phosphine ligand 32 as a colorless oily product (407 mg, 81%).

NMR data of bidentate phosphine ligand 32:

¹H NMR (400 MHz, CDCl₃): δ=7.15 (dd, J=6.0, 4.4 Hz, 1H), 7.06 (ddt, J=6.6, 4.6, 1.1 Hz, 1H), 3.07 (dd, J=13.7, 0.9 Hz, 2H), 2.41 (t, J=8.1 Hz, 4H), 1.58 (t, J=8.1 Hz, 4H), 1.13 (s, 9H).

¹³C NMR (100 MHz, CDCl₃): δ=211.53, 136.71, 131.51, 126.46, 38.82, 36.89, 33.51, 28.01, 23.58.

³¹P NMR (162 MHz, CDCl₃): δ=46.10.

### Preparation Example 33

### Preparation of Bidentate Phosphine Ligand 33: 1,2-Bis((di-tert-butylphosphino)methyl)-4-tert-butyl-benzene

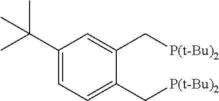

4-tert-Butyl-o-xylene (4.55 g, 28.1 mmol) (Aldrich) was diluted with heptane (100 ml), and NaOBu^{t} (8.1 g, 84.3 mmol), TMEDA (12.6 ml, 84.3 mmol) and BuⁿLi (2.5 M in hexane, 33.7 ml, 84.3 mmol) were added thereto. Butyl lithium was added dropwise and produced an immediate color change from colorless to yellow to orange to dark red. Thereafter, the solution was heated at 65° C. for 3 h to afford a brown/orange suspension.

The suspension was cooled at room temperature and the supernatant was removed by cannula. The brown precipitate residue was then washed with pentane (100 ml). Thereafter, the pentane wash was removed by cannula. The solid residue was then suspended in pentane (100 ml) and cooled in a cold water bath. But₂PCl (7.5 ml, 39.3 mmol) was added dropwise to the suspension. The resulting suspension was then stirred for 3 h and left to stand overnight.

Water (100 ml) was degassed with nitrogen for 30 min and then added to the suspension to afford a two-phase solution. The upper layer (organic phases) was diluted with pentane (100 ml) and the organic phases were collected via a cannula into a clean schlenk flask. The pentane extract was dried over sodium sulfate and transferred into a clean schlenk flask via a cannula. Afterwards, the solvent was removed under vacuum to afford orange oil. Methanol (100 ml) was added thereto to afford a two-phase solution. The solution was then heated to reflux (70° C.) to produce a light yellow solution and some colorless insoluble matters. Next, the solution was cooled at room temperature and filtered into a clean schlenk flask. Subsequently, the solution was placed in a freezer at −20° C. overnight, and produced off-white solid sediment. The remaining methanol solution was then removed by a cannula and the solid was dried under vacuum. The solid was separated in a glove box to afford bidentate phosphine ligand 31 (4.20 g, yield 33%).

### Preparation Example 34

### Preparation of Bidentate Phosphine Ligand 34: 1,2-Bis((di-tert-butylphosphino)methyl)benzene

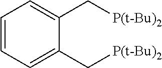

The ligand was prepared by the same method as described in Preparation Example 31, except for replacing 4-tert-butyl-o-xylene with an equimolar amount of o-xylene, to afford bidentate phosphine ligand 32 as a white solid (4.5 g, yield 40%).

### Example 1

1000 g of (31.25 mol) of methanol, 91.57 mg (0.10 mmol) tris(dibenzylideneacetone) dipalladium, 0.77 g (8 mmol) of methanesulfonic acid, and bidentate phosphine ligand 1 (167.21 mg, 0.50 mmol) were added to a 2-L autoclave and the autoclave was sealed. Ethylene and carbon monoxide at a molar ratio of 4:1 were introduced into this autoclave at a CO flow rate of 1.2 L/min, and reacted at a stirring speed of 500 r/min, a reaction pressure of 1.2 MPa, and a reaction temperature of 60° C. for a reaction time of 120 min.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 2

The reaction was carried out by the same method as described in Example 1, except that 91.57 mg of tris(dibenzylideneacetone) dipalladium was replaced with 42.28 mg (0.20 mmol) of dichlorodiammine palladium, the mass of methanol was changed to 500 g (15.63 mmol), and 291.35 mg (0.50 mmol) of bidentate phosphine ligand 2 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 3

The reaction was carried out by the same method as described in Example 1, except that 91.57 mg of tris(dibenzylideneacetone) dipalladium was preplaced with 46.50 mg (0.20 mmol) of diaminedinitritopalladium (II), the mass of methanol was changed to 500 g (15.63 mmol), and 187.20 mg (0.50 mmol) of bidentate phosphine ligand 3 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 4

The reaction was carried out by the same method as described in Example 1, except that 0.77 g of methanesulfonic acid was replaced with 1.27 g (8 mmol) of benzenesulfonic acid and 237.26 mg (0.50 mmol) of bidentate phosphine ligand 4 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 5

The reaction was carried out by the same method as described in Example 1, except that 0.77 g of methanesulfonic acid was replaced with 1.38 g (8 mmol) of p-toluenesulfonic acid and 235.25 mg (0.50 mmol) of bidentate phosphine ligand 5 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 6

The reaction was carried out by the same method as described in Example 1, except that the reaction temperature was change to 80° C. and 176.18 mg (0.50 mmol) of bidentate phosphine ligand 6 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 7

The reaction was carried out by the same method as described in Example 1, except that the reaction temperature was change to 100° C. and 177.17 mg of (0.50 mmol) of bidentate phosphine ligand 7 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 8

The reaction was carried out by the same method as described in Example 1, except that 91.57 mg of tris(dibenzylideneacetone) dipalladium was replaced with 49.31 mg (0.10 mmol) of [Rh(COD)Cl]₂, the mass of methanol was changed to 500 g (15.63 mmol), and 188.19 mg (0.50 mmol) of bidentate phosphine ligand 8 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 9

The reaction was carried out by the same method as described in Example 1, except that 91.57 mg of tris(dibenzylideneacetone) dipalladium was replaced with 67.17 mg (0.10 mmol) of [Ir(COD)Cl]₂, the mass of methanol was changed to 500 g (15.63 mmol), and 327.47 mg (0.50 mmol) of bidentate phosphine ligand 9 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 10

The reaction was carried out by the same method as described in Example 1, except that 209.25 mg (0.50 mmol) of bidentate phosphine ligand 10 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 11

The reaction was carried out by the same method as described in Example 1, except that 211.22 mg (0.50 mmol) of bidentate phosphine ligand 11 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 12

The reaction was carried out by the same method as described in Example 1, except that 210.24 mg (0.50 mmol) of bidentate phosphine ligand 12 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 13

The reaction was carried out by the same method as described in Example 1, except that 239.27 mg (0.50 mmol) of bidentate phosphine ligand 13 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 14

The reaction was carried out by the same method as described in Example 7, except that 335.37 mg (0.50 mmol) of bidentate phosphine ligand 14 was used and that ethylene and carbon monoxide at a molar ratio of 4:1 was changed to 1-hexene and carbon monoxide at the same molar ratio.

An appropriate amount of the reaction mixture was weighed and subjected to GC chromatography analysis. The conversion rate and selectivity as well as the yield of the product methyl enanthate were calculated (on a carbon monoxide basis), and the results were shown in Table 1.

### Example 15

The reaction was carried out by the same method as described in Example 7, except that 315.29 mg (0.50 mmol) of bidentate phosphine ligand 15 was used and that ethylene and carbon monoxide at a molar ratio of 4:1 was changed to 1-pentene and carbon monoxide at the same molar ratio.

An appropriate amount of the reaction mixture was weighed and subjected to GC chromatography analysis. The conversion rate and selectivity as well as the yield of the product methyl caproate were calculated (on a carbon monoxide basis), and the results were shown in Table 1.

### Example 16

The reaction was carried out by the same method as described in Example 7, except that 313.32 mg (0.50 mmol) of bidentate phosphine ligand 16 was used and that ethylene and carbon monoxide at a molar ratio of 4:1 was changed to 3-methyl-1-butene and carbon monoxide at the same molar ratio.

An appropriate amount of the reaction mixture was weighed and subjected to GC chromatography analysis. The conversion rate and selectivity as well as the yield of the product methyl 3-methylpentanoate were calculated (on a carbon monoxide basis), and the results were shown in Table 1.

### Example 17

The reaction was carried out by the same method as described in Example 7, except that 337.34 mg (0.50 mmol) of bidentate phosphine ligand 17 was used and that ethylene and carbon monoxide at a molar ratio of 4:1 was changed to 3,3-dimethyl-1-butene and carbon monoxide at the same molar ratio.

An appropriate amount of the reaction mixture was weighed and subjected to GC chromatography analysis. The conversion rate and selectivity as well as the yield of the product methyl 3,3-dimethylpentanoate were calculated (on a carbon monoxide basis), and the results were shown in Table 1.

### Example 18

The reaction was carried out by the same method as described in Example 7, except that 347.46 mg (0.50 mmol) of bidentate phosphine ligand 18 was used and that ethylene and carbon monoxide at a molar ratio of 4:1 was changed to styrene and carbon monoxide at the same molar ratio.

An appropriate amount of the reaction mixture was weighed and subjected to GC chromatography analysis. The conversion rate and selectivity as well as the yield of the product methyl 3-phenylpropionate were calculated (on a carbon monoxide basis), and the results were shown in Table 1.

### Example 19

The reaction was carried out by the same method as described in Example 7, except that 238.29 mg (0.50 mmol) of bidentate phosphine ligand 19 was used and that ethylene and carbon monoxide at a molar ratio of 4:1 was changed to 2-butene and carbon monoxide at the same molar ratio.

An appropriate amount of the reaction mixture was weighed and subjected to GC chromatography analysis. The conversion rate and selectivity as well as the yield of the product methyl 2-methylbutyrate were calculated (on a carbon monoxide basis), and the results were shown in Table 1.

### Example 20

The reaction was carried out by the same method as described in Example 7, except that 195.22 mg (0.50 mmol) of bidentate phosphine ligand 20 was used and that ethylene and carbon monoxide at a molar ratio of 4:1 was changed to cyclohexene and carbon monoxide at the same molar ratio.

An appropriate amount of the reaction mixture was weighed and subjected to GC chromatography analysis. The conversion rate and selectivity as well as the yield of the product methyl cyclohexanecarboxylate were calculated (on a carbon monoxide basis), and the results were shown in Table 1.

### Example 21

The reaction was carried out by the same method as described in Example 7, except that 253.30 mg (0.50 mmol) of bidentate phosphine ligand 21 was used and that ethylene and carbon monoxide at a molar ratio of 4:1 was changed to 3-hexene and carbon monoxide at the same molar ratio.

An appropriate amount of the reaction mixture was weighed and subjected to GC chromatography analysis. The conversion rate and selectivity as well as the yield of the product methyl 2-ethylpentanoate were calculated (on a carbon monoxide basis), and the results were shown in Table 1.

### Example 22

The reaction was carried out by the same method as described in Example 1, except that 252.32 mg (0.50 mmol) of bidentate phosphine ligand 22 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 23

The reaction was carried out by the same method as described in Example 1, except that 208.05 mg (0.50 mmol) of bidentate phosphine ligand 23 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 24

The reaction was carried out by the same method as described in Example 1, except that 296.95 mg (0.50 mmol) of bidentate phosphine ligand 24 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 25

The reaction was carried out by the same method as described in Example 1, except that 141.13 mg (0.50 mmol) of bidentate phosphine ligand 25 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 26

The reaction was carried out by the same method as described in Example 1, except that 154.17 mg (0.50 mmol) of bidentate phosphine ligand 26 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 27

The reaction was carried out by the same method as described in Example 1, except that 155.16 mg (0.50 mmol) of bidentate phosphine ligand 27 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 28

The reaction was carried out by the same method as described in Example 1, except that 168.20 mg (0.50 mmol) of bidentate phosphine ligand 28 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 29

The reaction was carried out by the same method as described in Example 1, except that 157.13 mg (0.50 mmol) of bidentate phosphine ligand 29 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 30

The reaction was carried out by the same method as described in Example 1, except that 183.21 mg (0.50 mmol) of bidentate phosphine ligand 30 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 31

The reaction was carried out by the same method as described in Example 1, except that 237.30 mg (0.50 mmol) of bidentate phosphine ligand 31 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 32

The reaction was carried out by the same method as described in Example 1, except that 251.33 mg (0.50 mmol) of bidentate phosphine ligand 32 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 33

The reaction was carried out by the same method as described in Example 1, except that 177.06 mg (0.37 mmol) of bidentate phosphine ligand 13, 67.76 mg (0.07 mmol) of tris(dibenzylideneacetone) dipalladium, and 0.57 g (5.92 mmol) of methanesulfonic acid were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 34

The reaction was carried out by the same method as described in Example 1, except that 670.73 mg (1.00 mmol) of bidentate phosphine ligand 14, 183.15 mg (0.20 mmol) of tris(dibenzylideneacetone) dipalladium, and 1.54 g (16 mmol) of methanesulfonic acid were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 35

The reaction was carried out by the same method as described in Example 1, except that 187.44 mg (0.37 mmol) of bidentate phosphine ligand 21, 67.76 mg (0.07 mmol) of tris(dibenzylideneacetone) dipalladium, and 0.57 g (5.92 mmol) of methanesulfonic acid.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 36

The reaction was carried out by the same method as described in Example 1, except that 957.08 mg (2.00 mmol) of bidentate phosphine ligand 13 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 37

The reaction was carried out by the same method as described in Example 1, except that 239.27 mg (0.50 mmol) of bidentate phosphine ligand 13 and 0.04 g (0.4 mmol) of methanesulfonic acid were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 38

The reaction was carried out by the same method as described in Example 1, except that 239.27 mg (0.50 mmol) of bidentate phosphine ligand 13 and 1.92 g (20 mmol) of methanesulfonic acid were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 39

The reaction was carried out by the same method as described in Example 1, except that 239.27 mg (0.50 mmol) of bidentate phosphine ligand 13, 0.96 g (10 mmol) of methanesulfonic acid, and 50 g (1.56 mol) of methanol were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 40

The reaction was carried out by the same method as described in Example 1, except that 133.99 mg (0.28 mmol) of bidentate phosphine ligand 13, 51.28 mg (0.06 mmol) of tris(dibenzylideneacetone) dipalladium, and 0.43 g (4.48 mmol) of methanesulfonic acid were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 41

The reaction was carried out by the same method as described in Example 1, except that 506.59 mg (1.00 mmol) of bidentate phosphine ligand 21 and 0.19 g (2 mmol) of methanesulfonic acid were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 42

The reaction was carried out by the same method as described in Example 1, except that 506.59 mg (1.00 mmol) of bidentate phosphine ligand 21 and 1.92 g (20 mmol) of methanesulfonic acid were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Example 43

The reaction was carried out by the same method as described in Example 1, except that 506.59 mg (1.00 mmol) of bidentate phosphine ligand 21 and 0.96 g (10 mmol) of methanesulfonic acid were used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Comparative Example 1

The reaction was carried out by the same method as described in Example 1, except that the amount of tris(dibenzylideneacetone) dipalladium was changed to 183.15 mg (0.20 mmol), the amount of methanesulfonic acid was changed to 1.54 g (16 mmol), and 450.66 mg (1.00 mmol) of bidentate phosphine ligand 33 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

### Comparative Example 2

The reaction was carried out by the same method as described in Example 1, except that the amount of tris(dibenzylideneacetone) dipalladium was changed to 183.15 mg (0.20 mmol), the amount of methanesulfonic acid was changed to 1.54 g (16 mmol), and 394.55 mg (1.00 mmol) of bidentate phosphine ligand 34 was used.

An appropriate amount of the reaction mixture was weighed and subjected to GC chromatography analysis. The conversion rate and selectivity as well as the yield of the product methyl propionate were calculated (on a carbon monoxide basis), and the results were shown in Table 1.

TABLE 1

| | | | Ligand | Con- | Selec- | |
|---|---|---|---|---|---|---|
| | Ligand | Structure of Bidentate | Amount/ | version | tivity/ | Yield/ |
| | No. | Phosphine Ligand | mg (mmol) | Rate/% | % | % |
| Example 1 | 1 | 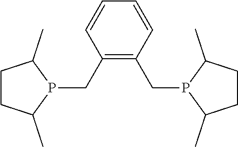 [SMILES: CC1CCC(C)P1Cc1ccccc1CP1C(C)CCC1C] | 167.21(0.50) | 96.7 | 97.3 | 94.09 |
| Example 2 | 2 | 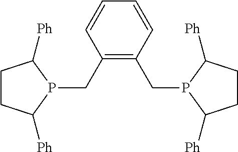 [SMILES: c1ccc(C2CCC(c3ccccc3)P2Cc2ccccc2CP2C(c3ccccc3)CCC2c2ccccc2)cc1] | 291.35(0.50) | 96.5 | 97.2 | 93.80 |
| Example 3 | 3 | 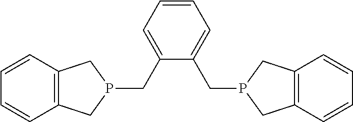 [SMILES: c1ccc2c(c1)CP(Cc1ccccc1CP1Cc3ccccc3C1)C2] | 187.20(0.50) | 96.2 | 96.7 | 93.03 |
| Example 4 | 4 | 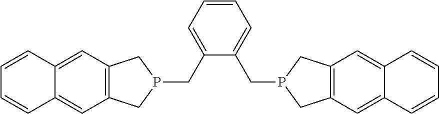 [SMILES: c1ccc(CP2Cc3cc4ccccc4cc3C2)c(CP2Cc3cc4ccccc4cc3C2)c1] | 237.26(0.50) | 96.3 | 96.6 | 93.03 |
| Example 5 | 5 | 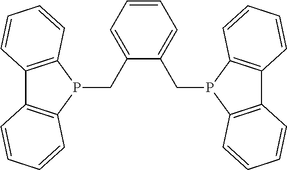 [SMILES: c1ccc(Cp2c3ccccc3c3ccccc32)c(Cp2c3ccccc3c3ccccc32)c1] | 235.25(0.50) | 96.1 | 96.7 | 92.93 |
| Example 6 | 6 | 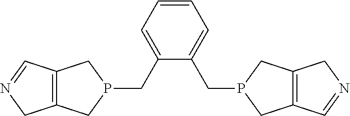 [SMILES: C1=NCC2=C1CP(Cc1ccccc1CP1CC3=C(CN=C3)C1)C2] | 176.18(0.50) | 96.3 | 96.9 | 93.31 |
| Example 7 | 7 | 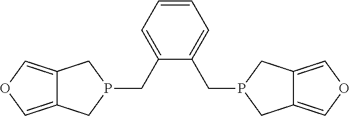 [SMILES: c1ccc(CP2Cc3cocc3C2)c(CP2Cc3cocc3C2)c1] | 177.17(0.50) | 96.4 | 97.1 | 93.60 |
| Example 8 | 8 | 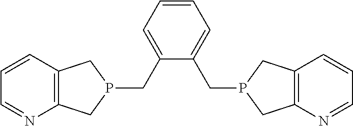 [SMILES: c1ccc(CP2Cc3cccnc3C2)c(CP2Cc3cccnc3C2)c1] | 188.19(0.50) | 96.2 | 96.8 | 93.12 |
| Example 9 | 9 | 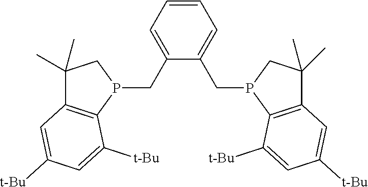 [SMILES: CC(C)(C)c1cc(C(C)(C)C)c2c(c1)C(C)(C)CP2Cc1ccccc1CP1CC(C)(C)c2cc(C(C)(C)C)cc(C(C)(C)C)c21] | 327.47(0.50) | 97.1 | 96.8 | 93.99 |
| Example 10 | 10 | 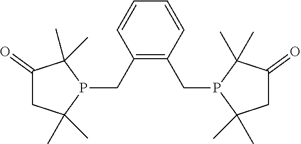 [SMILES: CC1(C)CC(=O)C(C)(C)P1Cc1ccccc1CP1C(C)(C)CC(=O)C1(C)C] | 209.25(0.50) | 96.8 | 97.0 | 93.90 |
| Example 11 | 11 | 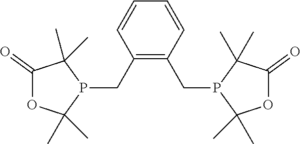 [SMILES: CC1(C)OC(=O)C(C)(C)P1Cc1ccccc1CP1C(C)(C)OC(=O)C1(C)C] | 211.22(0.50) | 98.5 | 98.7 | 97.22 |
| Example 12 | 12 | 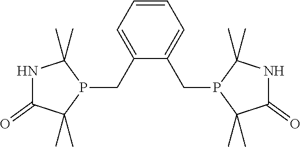 [SMILES: CC1(C)NC(=O)C(C)(C)P1Cc1ccccc1CP1C(C)(C)NC(=O)C1(C)C] | 210.24(0.50) | 97.8 | 98.0 | 95.84 |
| Example 13 | 13 | 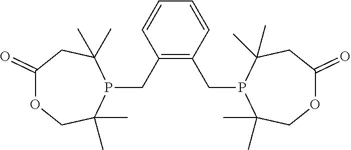 [SMILES: CC1(C)COC(=O)CC(C)(C)P1Cc1ccccc1CP1C(C)(C)COC(=O)CC1(C)C] | 239.27(0.50) | 98.8 | 99.2 | 98.01 |
| Example 14 | 14 | 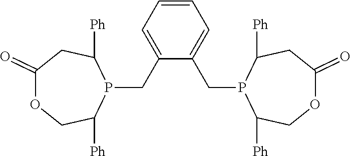 [SMILES: O=C1CC(c2ccccc2)P(Cc2ccccc2CP2C(c3ccccc3)COC(=O)CC2c2ccccc2)C(c2ccccc2)CO1] | 335.37(0.50) | 98.6 | 99.0 (n/iso = 99:1) | 97.61 |
| Example 15 | 15 | 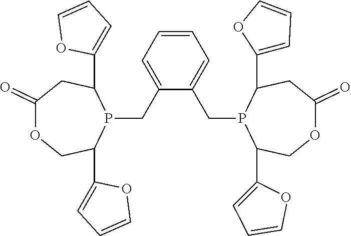 [SMILES: O=C1CC(c2ccco2)P(Cc2ccccc2CP2C(c3ccco3)COC(=O)CC2c2ccco2)C(c2ccco2)CO1] | 315.29(0.50) | 98.3 | 98.5 (n/iso = 97:3) | 96.83 |
| Example 16 | 16 | 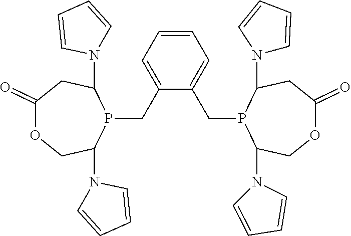 [SMILES: O=C1CC(n2cccc2)P(Cc2ccccc2CP2C(n3cccc3)COC(=O)CC2n2cccc2)C(n2cccc2)CO1] | 313.32(0.50) | 98.2 | 98.6 (n/iso = 97:3) | 96.83 |
| Example 17 | 17 | 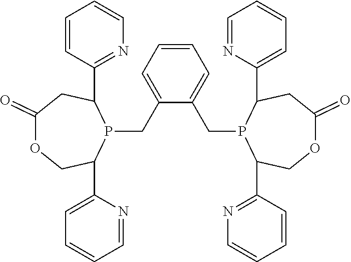 [SMILES: O=C1CC(c2ccccn2)P(Cc2ccccc2CP2C(c3ccccn3)COC(=O)CC2c2ccccn2)C(c2ccccn2)CO1] | 337.34(0.50) | 98.5 | 98.7 (n/iso = 98:2) | 97.22 |
| Example 18 | 18 | 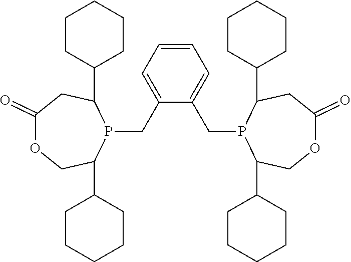 [SMILES: O=C1CC(C2CCCCC2)P(Cc2ccccc2CP2C(C3CCCCC3)COC(=O)CC2C2CCCCC2)C(C2CCCCC2)CO1] | 347.46(0.50) | 98.2 | 98.4 (n/iso = 97:3) | 96.63 |
| Example 19 | 19 | 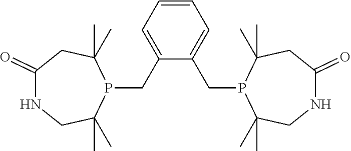 [SMILES: CC1(C)CNC(=O)CC(C)(C)P1Cc1ccccc1CP1C(C)(C)CNC(=O)CC1(C)C] | 238.29(0.50) | 97.8 | 98.1 | 95.94 |
| Example 20 | 20 | 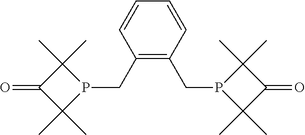 [SMILES: CC1(C)C(=O)C(C)(C)P1Cc1ccccc1CP1C(C)(C)C(=O)C1(C)C] | 195.22(0.50) | 96.7 | 96.8 | 93.61 |
| Example 21 | 21 | 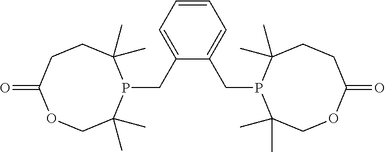 [SMILES: CC1(C)CCC(=O)OCC(C)(C)P1Cc1ccccc1CP1C(C)(C)CCC(=O)OCC1(C)C] | 253.30(0.50) | 98.7 | 99.3 | 98.01 |
| Example 22 | 22 | 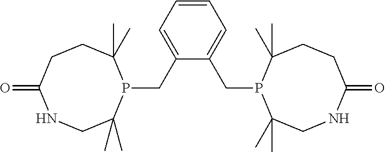 [SMILES: CC1(C)CCC(=O)NCC(C)(C)P1Cc1ccccc1CP1C(C)(C)CCC(=O)NCC1(C)C] | 252.32(0.50) | 97.9 | 98.0 | 95.94 |
| Example 23 | 23 | 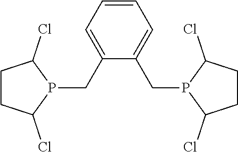 [SMILES: ClC1CCC(Cl)P1Cc1ccccc1CP1C(Cl)CCC1Cl] | 208.05(0.50) | 97.7 | 97.8 | 95.55 |
| Example 24 | 24 | 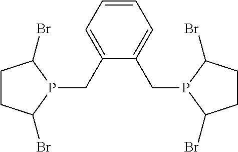 [SMILES: BrC1CCC(Br)P1Cc1ccccc1CP1C(Br)CCC1Br] | 296.95(0.50) | 97.6 | 98.0 | 95.65 |
| Example 25 | 25 | 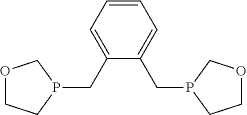 [SMILES: c1ccc(CP2CCOC2)c(CP2CCOC2)c1] | 141.13(0.50) | 95.8 | 96.7 | 92.64 |
| Example 26 | 26 | 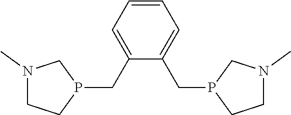 | 154.17(0.50) | 95.5 | 97.0 | 92.64 |
| Example 27 | 27 | 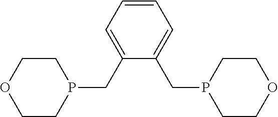 [SMILES: c1ccc(CP2CCOCC2)c(CP2CCOCC2)c1] | 155.16(0.50) | 96.0 | 96.8 | 92.93 |
| Example 28 | 28 | 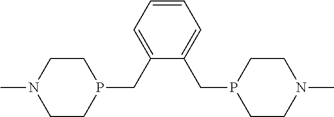 | 168.20(0.50) | 95.9 | 96.6 | 92.64 |
| Example 29 | 29 | 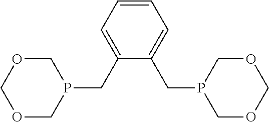 [SMILES: c1ccc(CP2COCOC2)c(CP2COCOC2)c1] | 157.13(0.50) | 95.5 | 96.4 | 92.06 |
| Example 30 | 30 | 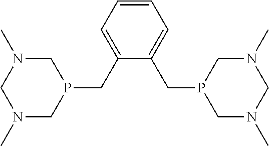 [SMILES: CN1CN(C)CP(Cc2ccccc2CP2CN(C)CN(C)C2)C1] | 183.21(0.50) | 95.4 | 96.5 | 92.06 |
| Example 31 | 31 | 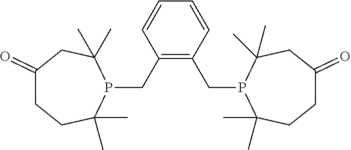 [SMILES: CC1(C)CCC(=O)CC(C)(C)P1Cc1ccccc1CP1C(C)(C)CCC(=O)CC1(C)C] | 237.30(0.50) | 97.1 | 97.3 | 94.48 |
| Example 32 | 32 | 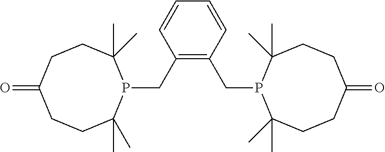 [SMILES: CC1(C)CCC(=O)CCC(C)(C)P1Cc1ccccc1CP1C(C)(C)CCC(=O)CCC1(C)C] | 251.33(0.50) | 97.4 | 97.2 | 94.67 |
| Example 33 | 13 | 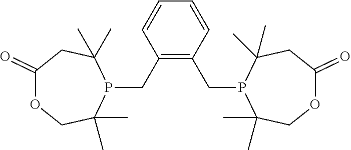 [SMILES: CC1(C)COC(=O)CC(C)(C)P1Cc1ccccc1CP1C(C)(C)COC(=O)CC1(C)C] | 177.06(0.37) | 98.5 | 99.0 | 97.52 |
| Example 34 | 14 | 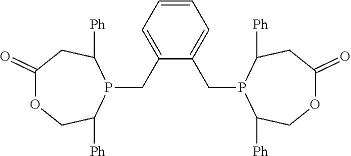 [SMILES: O=C1CC(c2ccccc2)P(Cc2ccccc2CP2C(c3ccccc3)COC(=O)CC2c2ccccc2)C(c2ccccc2)CO1] | 670.73(1.00) | 98.7 | 99.1 | 97.81 |
| Example 35 | 21 | 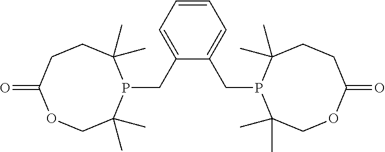 [SMILES: CC1(C)CCC(=O)OCC(C)(C)P1Cc1ccccc1CP1C(C)(C)CCC(=O)OCC1(C)C] | 187.44(0.37) | 98.3 | 99.0 | 97.32 |
| Example 36 | 13 | 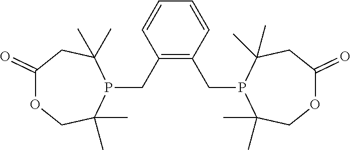 [SMILES: CC1(C)COC(=O)CC(C)(C)P1Cc1ccccc1CP1C(C)(C)COC(=O)CC1(C)C] | 957.08(2.00) | 98.1 | 98.8 | 96.92 |
| Example 37 | 13 | 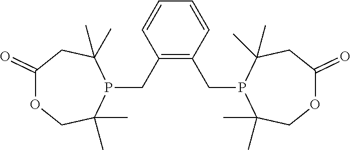 [SMILES: CC1(C)COC(=O)CC(C)(C)P1Cc1ccccc1CP1C(C)(C)COC(=O)CC1(C)C] | 239.27(0.50) | 97.6 | 96.3 | 93.99 |
| Example 38 | 13 | 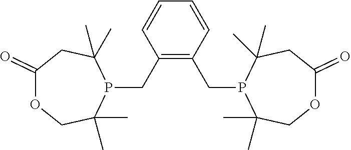 [SMILES: CC1(C)COC(=O)CC(C)(C)P1Cc1ccccc1CP1C(C)(C)COC(=O)CC1(C)C] | 239.27(0.50) | 97.8 | 97.5 | 95.36 |
| Example 39 | 13 | 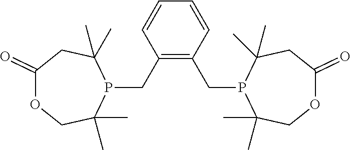 [SMILES: CC1(C)COC(=O)CC(C)(C)P1Cc1ccccc1CP1C(C)(C)COC(=O)CC1(C)C] | 239.27(0.50) | 95.2 | 97.8 | 93.11 |
| Example 40 | 13 | 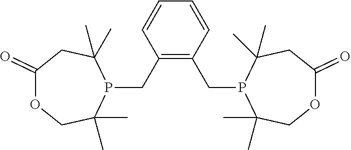 [SMILES: CC1(C)COC(=O)CC(C)(C)P1Cc1ccccc1CP1C(C)(C)COC(=O)CC1(C)C] | 133.99(0.28) | 97.9 | 98.7 | 96.63 |
| Example 41 | 21 | 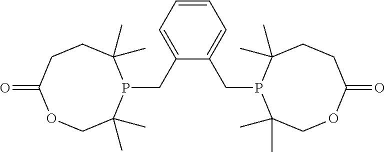 [SMILES: CC1(C)CCC(=O)OCC(C)(C)P1Cc1ccccc1CP1C(C)(C)CCC(=O)OCC1(C)C] | 506.59(1.00) | 97.1 | 97.2 | 94.38 |
| Example 42 | 21 | 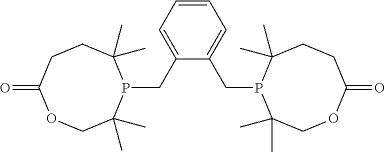 [SMILES: CC1(C)CCC(=O)OCC(C)(C)P1Cc1ccccc1CP1C(C)(C)CCC(=O)OCC1(C)C] | 506.59(1.00) | 97.4 | 97.6 | 95.06 |
| Example 43 | 21 | 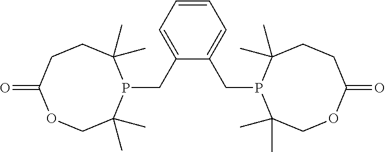 [SMILES: CC1(C)CCC(=O)OCC(C)(C)P1Cc1ccccc1CP1C(C)(C)CCC(=O)OCC1(C)C] | 506.59(1.00) | 98.4 | 98.7 | 97.12 |
| Comparative Example 1 | 33 | 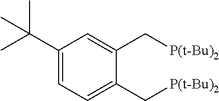 | 450.66(1.00) | 93.7 | 94.5 | 88.55 |
| Comparative Example 2 | 34 | 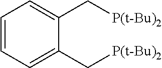 | 394.55(1.00) | 93.9 | 94.8 | 89.02 |

As shown in Table 1, Examples 1 to 43, in which a catalyst system containing a specific bidentate phosphine ligand was used in the method for the olefin carbonylation reaction provided herein, show that the bidentate phosphine ligand and the compound of a Group VIII metal are used in smaller amounts, the selectivity and yield of the product are higher, and the conversion rate of the substrate is higher, as compared to Comparative Examples 1 and 2 in which a catalyst system containing an additional bidentate phosphine ligand is used. Among them, Examples 11, 12 to 19, 21, 22, and 33 to 43, in which bidentate phosphine ligands containing a lactone group or a lactam group as a monocyclic group are used in the catalyst systems, show further higher selectivity and yield; and Examples 11, 13 to 18, 21, 33 to 36, 40, and 43, in which the bidentate phosphine ligands containing a lactone group as a monocyclic group are used, show the highest selectivity and yield of the product.

What is claimed is:

1. A catalyst system for a carbonylation reaction, comprising the following components:
(a) a Group VIII metal or a compound of a Group VIII metal;
(b) a bidentate phosphine ligand; and
(c) an acidic additive;
wherein the bidentate phosphine ligand as component (b) is represented by the following formula (I): 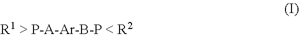
wherein P represents a trivalent phosphorus atom;
Ar represents an aromatic group, A and B each represent a single bond or alkylene, and A and B are located at ortho positions on the Ar group;
each of R¹ and R², together with the P atom attached thereto, form a monocyclic group that is a phospha monocyclic group having at least 3 carbon atoms on the ring;
the monocyclic group is selected from following groups with or without a substituent: an alicyclic group, a lactone group, a lactam group, a cyclic ketone group, a heterocyclic group containing at least one N atom, or a heterocyclic group containing at least one O atom; the substituent is alkyl, cycloalkyl, substituted or unsubstituted aryl, a halogen atom, a nitrogen-containing group or an oxygen-containing group; and the substituent is attached to the monocyclic group via a single bond or by sharing a plurality of carbon atoms.

2. The catalyst system according to claim 1, wherein
A and B each represent C1-C6 alkylene;
Ar represents phenyl or naphthyl;
the monocyclic group is a monocyclic group having 3 to 10 carbon atoms on the ring, and the substituent is alkyl, cycloalkyl, substituted or unsubstituted aryl, a halogen atom, a nitrogen-containing group, or an oxygen-containing group.

3. The catalyst system according to claim 1, wherein
the monocyclic group is selected from following groups with or without a substituent: a phosphacyclopentanyl group, a phosphapropiolactone group, a phosphabutyrolactone group, a phosphavalerolactone group, a phosphacaprolactone group, a phosphaheptanolactone group, a phosphacyclobutanone group, a phosphacyclopentanone group, a phosphacyclohexanone group, a phosphacycloheptanone group, a phosphacyclooctanone group, a phosphatetrahydrofuranyl group, a phosphatetrahydropyrrolyl group, a phosphatetrahydropyranyl group, a phosphahexahydropyridinyl group, a phosphahexahydropyrimidinyl group, a dioxaphosphacyclohexane group, a phosphapropionam group, a phosphabutyrolactam group, a phosphavalerolactam group, a phosphacaprolactam group, or a phosphaheptanolactam group; and
the substituent is alkyl having 1 to 5 carbon atoms, substituted or unsubstituted phenyl, substituted or unsubstituted naphthyl, a chlorine atom, a bromine atom, cyclohexyl, furyl, pyrrolyl, pyridyl, or [2-H]-pyrrolyl.

4. The catalyst system according to claim 1, wherein
the bidentate phosphine ligand is one or more selected from the group consisting of:

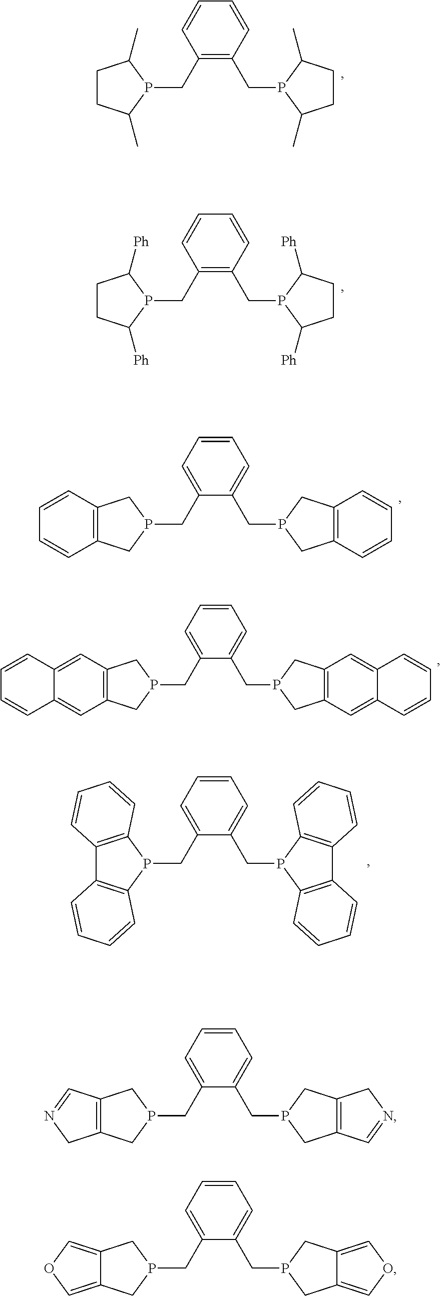

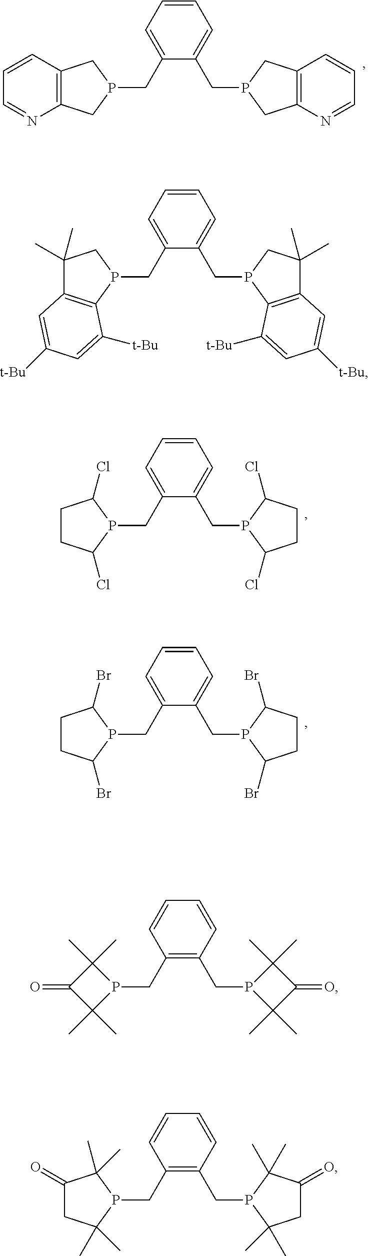 [SMILES: c1ccc(CP2Cc3cccnc3C2)c(CP2Cc3cccnc3C2)c1 ; CC(C)(C)c1cc(C(C)(C)C)c2c(c1)C(C)(C)CP2Cc1ccccc1CP1CC(C)(C)c2cc(C(C)(C)C)cc(C(C)(C)C)c21 ; ClC1CCC(Cl)P1Cc1ccccc1CP1C(Cl)CCC1Cl ; BrC1CCC(Br)P1Cc1ccccc1CP1C(Br)CCC1Br ; CC1(C)C(=O)C(C)(C)P1Cc1ccccc1CP1C(C)(C)C(=O)C1(C)C ; CC1(C)CC(=O)C(C)(C)P1Cc1ccccc1CP1C(C)(C)CC(=O)C1(C)C]

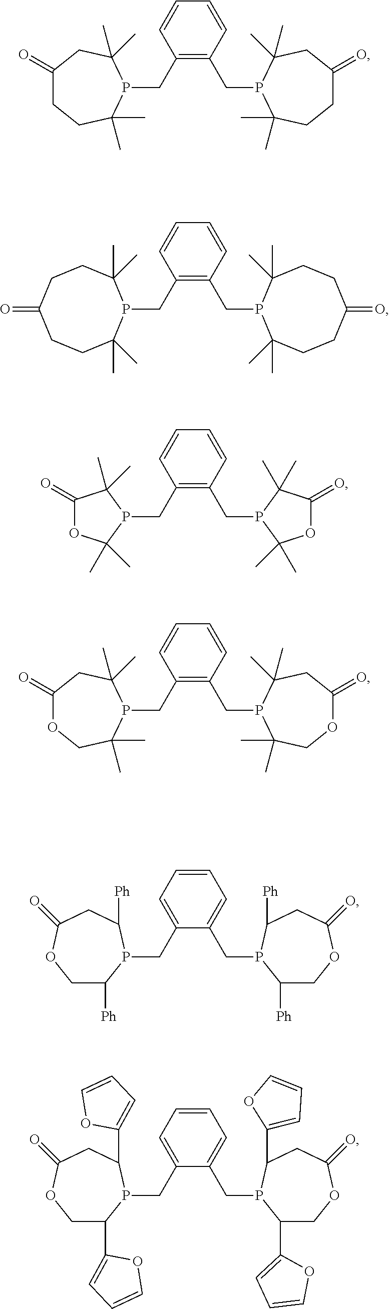 [SMILES: CC1(C)CCC(=O)CC(C)(C)P1Cc1ccccc1CP1C(C)(C)CCC(=O)CC1(C)C ; CC1(C)CCC(=O)CCC(C)(C)P1Cc1ccccc1CP1C(C)(C)CCC(=O)CCC1(C)C ; CC1(C)OC(=O)C(C)(C)P1Cc1ccccc1CP1C(C)(C)OC(=O)C1(C)C ; CC1(C)COC(=O)CC(C)(C)P1Cc1ccccc1CP1C(C)(C)COC(=O)CC1(C)C ; O=C1CC(c2ccccc2)P(Cc2ccccc2CP2C(c3ccccc3)COC(=O)CC2c2ccccc2)C(c2ccccc2)CO1 ; O=C1CC(c2ccco2)P(Cc2ccccc2CP2C(c3ccco3)COC(=O)CC2c2ccco2)C(c2ccco2)CO1]

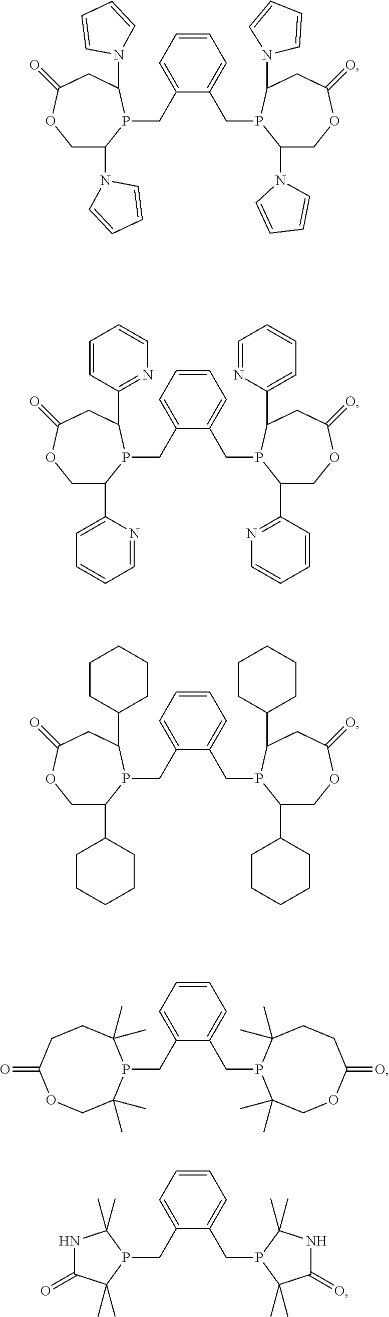 [SMILES: O=C1CC(n2cccc2)P(Cc2ccccc2CP2C(n3cccc3)COC(=O)CC2n2cccc2)C(n2cccc2)CO1 ; O=C1CC(c2ccccn2)P(Cc2ccccc2CP2C(c3ccccn3)COC(=O)CC2c2ccccn2)C(c2ccccn2)CO1 ; O=C1CC(C2CCCCC2)P(Cc2ccccc2CP2C(C3CCCCC3)COC(=O)CC2C2CCCCC2)C(C2CCCCC2)CO1 ; CC1(C)CCC(=O)OCC(C)(C)P1Cc1ccccc1CP1C(C)(C)CCC(=O)OCC1(C)C ; CC1(C)NC(=O)C(C)(C)P1Cc1ccccc1CP1C(C)(C)NC(=O)C1(C)C]

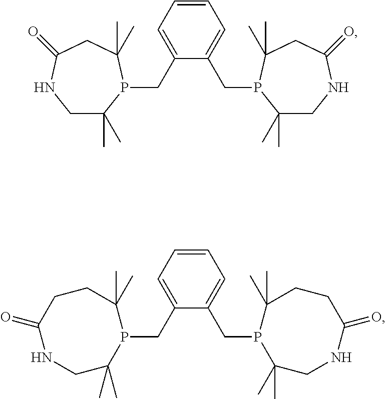 [SMILES: CC1(C)CNC(=O)CC(C)(C)P1Cc1ccccc1CP1C(C)(C)CNC(=O)CC1(C)C ; CC1(C)CCC(=O)NCC(C)(C)P1Cc1ccccc1CP1C(C)(C)CCC(=O)NCC1(C)C]

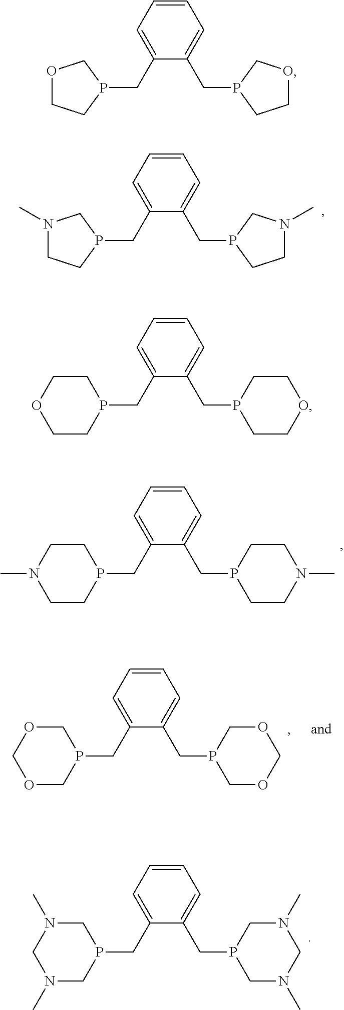

5. The catalyst system according to claim 1, wherein a molar ratio of
the component (b) to the component (a) is 2:1 to 10:1;
a molar ratio of the component (c) to the component (a) is 2:1 to 100:1.

6. The catalyst system according to claim 1, wherein
in the component (a):
the Group VIII metal comprises cobalt, nickel, palladium, rhodium, ruthenium, iridium or platinum;
the compound of the Group VIII metal comprises: a compound formed of the Group VIII metal and the following substance: sulfuric acid, nitric acid, phosphoric acid, formic acid, acetic acid, propionic acid, trichloroacetic acid, trifluoroacetic acid, methanesulfonic acid, chlorosulfonic acid, fluorosulfonic acid, trifluoromethanesulfonic acid, benzenesulfonic acid, naphthalenesulfonic acid, toluenesulfonic acid, sulfonated ion exchange resin, or perhalogenic acid; or a complex of zero-valent palladium, rhodium, iridium, platinum, or ruthenium;
the acidic additive as the component (c) is an acid having a pKa value of less than 5 in an aqueous solution at 25° C.

7. The catalyst system according to claim 1, wherein
the acidic additive as the component (c) comprises at least one of methanesulfonic acid, trifluoromethanesulfonic acid, tert-butylsulfonic acid, p-toluenesulfonic acid, 2-hydroxypropyl-2-sulfonic acid, 2,4,6-trimethylmethanesulfonic acid, perchloric acid, phosphoric acid, methylphosphoric acid, and sulfuric acid.

8. A method for a carbonylation reaction, the method comprising reacting an unsaturated compound having a double bond with carbon monoxide and an alcohol in the presence of a catalyst system;
wherein the catalyst system is the catalyst system according to claim 5.

9. The method according to claim 8, wherein a molar ratio of the unsaturated compound having a double bond to the carbon monoxide is 1:1 to 100:1;
a molar ratio of the unsaturated compound having a double bond to the component (a) in the catalyst system is 50:1 to 600:1;
a mass ratio of the alcohol to the component (a) in the catalyst system is 500:1 to 20000:1.

10. The method according to claim 8, wherein the reaction is conducted under the following operating conditions:
a reaction pressure is at 1 to 20 MPa; and a reaction temperature is at 50 to 200° C.

11. The method according to claim 8, wherein
the unsaturated compound having a double bond is a substituted or unsubstituted C2-C20 olefin; when the olefin has a substituent, the substituent is C1-C10 alkyl, C6-C12 aryl, C1-C4 alkoxy, halogen-substituted C6-C12 aryl, a C2-C6 ester group, or a nitrogen-containing heterocyclic group;
the alcohol is a substituted or unsubstituted linear or branched C1-C10 alkanol; when the alcohol is an alcohol having a substituent, the substituent is C1-C6 alkyl, C6-C20 aryl, C2-C10 heterocyclyl, halogen, cyano or nitro.

12. The catalyst system according to claim 1, wherein a molar ratio of the component (b) to the component (a) is 2:1 2:1 to 5:1;
a molar ratio of the component (c) to the component (a) is 10:1 to 50:1.

13. The method according to claim 8, wherein a molar ratio of the unsaturated compound having a double bond to the carbon monoxide is preferably 2:1 to 50:1;
a molar ratio of the unsaturated compound having a double bond to the component (a) in the catalyst system is 100:1 to 300:1;
a mass ratio of the alcohol to the component (a) in the catalyst system is 5000:1 to 15000:1.

14. The method according to claim 8, wherein the reaction is conducted under the following operating conditions:
a reaction pressure is at 1 to 10 MPa; and a reaction temperature is at 60 to 150° C.

15. The method according to claim 8, wherein
the unsaturated compound having a double bond is a substituted or unsubstituted C2-C16 olefin; when the olefin has a substituent, the substituent is C1-C10 alkyl, C6-C12 aryl, C1-C4 alkoxy, halogen-substituted C6-C12 aryl, a C2-C6 ester group, or a nitrogen-containing heterocyclic group;
the alcohol is a substituted or unsubstituted linear or branched C1-C10 alkanol; when the alcohol is an alcohol having a substituent, the substituent is C1-C6 alkyl or C6-C10 aryl.

16. The method according to claim 8, wherein
the unsaturated compound having a double bond is a C2-C6 olefin;
the alcohol is a C1-C6 monohydric alkanol.
